(19) United States
(12) Patent Application Publication
(10) Pub. No.: US 2014/0308597 A1
(43) Pub. Date: Oct. 16, 2014

(54) Steam Reforming Of Methanol

(71) Applicant: Shik Chi Edman Tsang, Oxford (GB)

(72) Inventor: Shik Chi Edman Tsang, Oxford (GB)

(73) Assignee: Isis Innovation Limited, Oxford (GB)

(21) Appl. No.: 14/130,991

(22) PCT Filed: Jul. 6, 2012
(86) PCT No.: PCT/GB2012/051601

§ 371 Date: Apr. 16, 2014

(30) Foreign Application Priority Data
Jul. 8, 2011 (GB) 1111781.9

(51) Int. Cl. C01B 3/32; B01J 23/825; H01M 8/06; C07C 29/154

(52) U.S. Cl. CPC C01B 3/326; C07C 29/154; B01J 23/825; H01M 8/0618
USPC 429/424; 518/713; 423/648.1; 502/342

(57) ABSTRACT

The invention provides a process for producing H₂ by steam reforming of methanol, which process comprises contacting a gas phase comprising (a) CH₃OH and (b) H₂0 with a solid catalyst, which solid catalyst comprises a mixed metal oxide, which mixed metal oxide comprises copper, zinc and gallium, wherein the atomic percentage of copper relative to the total number of metal atoms in the oxide is from 20 at. % to 55 at. %. The solid catalyst itself is also an aspect of the present invention, as is a process for producing the catalyst, which process comprises: (1) a co-precipitation step, comprising contacting: (a) a solution of copper nitrate, zinc nitrate and gallium nitrate, wherein the atomic percentage of copper relative to the total number of metal atoms in said solution is from 20 at. % to 55 at. %, with (b) a metal carbonate, to produce a co-precipitate comprising said copper, zinc and gallium; (2) a separation step, comprising separating the co-precipitate from solution; (3) a calcination step, comprising calcining the co-precipitate by heating the co-precipitate in air; and, optionally, (4) a reduction step, comprising heating the calcined product in the en presence of H₂. Further provided is the use of the catalyst of the invention in a process for producing H₂ by steam reforming of methanol. Additionally, the invention provides a fuel cell system comprising a fuel cell, such as a proton exchange membrane (PEM) fuel cell, and a methanol reformer comprising a catalyst of the invention. Portable electronic devices comprising a fuel cell system of the invention are also provided. A further aspect of the invention is the use of a catalyst of the invention in a process for producing methanol by the hydrogenation of carbon dioxide. Thus, the invention further provides a process for producing methanol by the hydrogenation of carbon dioxide, which process comprises contacting a gas phase comprising (a) C0₂ and (b) H₂, with a catalyst of the invention.

### FIELD OF THE INVENTION

The invention relates to a process for producing hydrogen by steam reforming of methanol, a catalyst for use in the process, and a process for producing the catalyst.

### BACKGROUND TO THE INVENTION

Global efforts are currently under way to minimize the emissions of NOx, SOx, hydrocarbons, CO, and CO₂. The use of hydrogen as an environmentally friendly energy carrier has been massively encouraged over the last years. Hydrogen is considered as the best fuel because of no emission of pollutants and also offers high efficiency when used in proton exchange membrane (PEM) fuel cells. Particularly, for portable applications such as cell phones, mp3-players, laptop computers and similar niche products, the use of PEM fuel cells is deemed to be more energy efficient than battery technology (Zhao, T. S. Microfuel cells: Principles and Applications, Elsevier, USA, 2009). Low temperature PEM fuel cells and micro fabrication technologies are potentially the preferred choices for these consumer products. There are a number of ways of obtaining hydrogen from both renewable and non-renewable sources on a large industrial scale, but the storage and transfer of hydrogen in solid systems for mobile use are problematic because of poor volumetric and weight energy densities (Van den Berg, A. W. C. & Arean, C. O. Chem. Commun, 668-681, 2008). In addition, ultra-pure hydrogen gas is required by the PEM fuel cells. In particular, the gas stream has to be free from CO gas (<10 ppm) otherwise the catalytic performance of the fuel cells is significantly degraded (Springer, T. E., Rockward, T., Zawodzinski, T. A. & Gottesfeld, S. J. Electrochem. Soc. 148, A11-A23, 2001). The use of on-board reforming of organic compounds with downstream multistage CO post-treatments such as the water gas shift (WGS) reaction, selective oxidation of CO to CO₂ (SELOX), hydrogenation of CO to methane or membrane technology, etc. is not applicable. This is because these cumbersome multistage processes commonly taken place at elevated temperatures, which precludes their adaptation in the small portable devices where space and heat management are at a premium.

Hydrogen stored in a chemical form as liquid organic compounds and released in-situ on demand at room temperature without CO contamination appears to be a more promising direction for mobile fuel cells. The primary liquid fuel can be stored in a disposable or recycled cartridge, which is changeable and logistically easily available. The production of hydrogen from organic compounds such as formic acid, which is nontoxic and a liquid at room temperature, with a density of 1.22 g. mL⁻³, has recently been demonstrated. The use of methanol, however, which is a key platform chemical for present fuel and chemical infrastructures with high energy content 5420 kcal kg⁻¹, is economically more attractive (Olah, G. A.; Coeppert, A.; Prakash, G. K. S. Beyond Oil and Gas: The Methanol Economy, Wiley-VCH, 2^{nd} Edit. Los Angeles, Calif., USA, 1998; WO 98/29333). Although methanol is currently produced industrially from non-renewable fuels over Cu/ZnO/Al₂O₃ catalysts (Toyir, J.; Ramirez de la Piscina, P.; Fierro, J. L. S.; Homs, N. Appl. Catal. B: Env. 29, 207-215, 2001; Saito, M.; Fujitani, T.; Takeuchi, M.; Watanabe, T. Appl. Catal. A: Gen. 138, 311-318, 1996; Sloczyn'ski, J.; Grabowski, R.; Olszewski, P.; Kozlowska, A. Stoch, J.; Lachowska, M.; Skrzypek, J. Appl. Catal. A: Gen. 310, 127-137 2006) in-situ hydrogen from methanol facilitates efficient on site energy conversion and cleaner emission until green methanol can be produced.

Low temperature steam reforming of methanol is seen as a promising route to hydrogen production. In particular, the direct, catalytic production of hydrogen by Non-Syngas Direct Steam Reforming (NSGDSR) of methanol is an attractive proposition. This route offers many advantages over other methods in terms of energy efficiency, CO mitigation and safety considerations. It is also in clear contrast with the conventional complex route involving steam reformation to syngas, followed by water gas shift and CO cleanup stages for the hydrogen production.

In PEM fuel cells, the on-board steam reforming process (Eq. 1) can provide a source of hydrogen in-situ, which is then combined downstream with oxygen to produce water (Eq. 2), with an accompanying release of energy:

CH₃OH+H₂O⇄3H₂+CO₂ ΔH=+49.7 kJ mol⁻¹ (Eq. 1)

H₂+½O₂⇄H₂O ΔH=−286 kJ mol⁻¹ (Eq. 2)

CO is considered to be formed via methanol decomposition (Eq. 3) and the reverse WGS reaction (Eq. 4):

CH₃OH⇄CO+2H₂ ΔH=+90.2 kJ mol⁻¹ (Eq. 3)

CO₂+H₂⇄CO+H₂O ΔH=+41.2 kJ mol⁻¹ (Eq. 4)

CO production must be minimised as much as possible, since levels greater than 10 ppm in the gas stream may poison the Pt-based catalyst used in the downstream reaction (Eq. 2) and severely impair its performance. A key challenge therefore is to provide a suitable steam-reforming catalyst that can operate at low temperatures (150-200° C.) and minimise CO production without the need for complicated downstream multi-stage CO post-treatment. The catalysts currently used for methanol steam reforming fall into two main categories: Cu-based and group 8-10 metals. Cu-based catalysts generally achieve higher activity but are unstable, being susceptible to deactivation over time due to thermal sintering, whereas group 8-10 metals provide greater stability at the expense of activity. The mechanism for CO production with Cu-based catalysts remains a controversial topic. It is not yet clear whether CO is produced primarily via methanol decomposition or the reverse WGS reaction. FT-IR studies with CuZnZrAlOx have shown that CO is formed as a secondary product via the reverse WGS reaction only, and not via methanol decomposition, but in general the matter is still far from settled. A key challenge therefore is to develop efficient catalysts for the production of hydrogen from steam-methanol reformation, in which the product gas stream contains a very low concentration (towards less than 10 ppm) of CO gas. Lachowska, M., Reac Kinet Mech Cat (2010) 101:85-91 describes a study of steam reformation of methanol over a mixed Cu/Zn/Zr/Ga oxide catalyst, which contains 65.3 wt. % CuO, 26.3 wt % ZnO, 4.5 wt. % ZrO₂ and 3.9 wt. % Ga₂O₃ (i.e. 67.2 at. % Cu, 26.4 at. % Zn, 3.0 at. % Zr and 3.4 at. % Ga, relative to the total number of metal atoms in the oxide). However, the study only provides calculated carbon monoxide concentrations presented at the parts per hundred (percent) level, indicating that the level of CO production has not been controlled at the parts per million (ppm) level. It remains an important challenge therefore to provide a suitable steam-reforming catalyst that can operate at low temperatures (150-200° C.) and minimise CO production at the ppm level, without the need for complicated downstream multi-stage CO post-treatments.

### SUMMARY OF THE INVENTION

The inventors have provided a heterogeneous catalyst which is active for the production of hydrogen by Non-Syngas Direct Steam Reforming (NSGDSR) of methanol at low temperatures and atmospheric pressure. Little or no CO is produced at the ppm level, making the NSGDSR process suitable for use in PEM fuel cell applications.

Accordingly, the invention provides a process for producing H₂ by steam reforming of methanol, which process comprises contacting a gas phase comprising (a) CH₃OH and (b) H₂O with a solid catalyst, which solid catalyst comprises a mixed metal oxide, which mixed metal oxide comprises copper, zinc and gallium, wherein the atomic percentage of copper relative to the total number of metal atoms in the oxide is from 20 at. % to 55 at. %.

In another aspect, the invention provides a catalyst for use in a process for producing H₂ by steam reforming of methanol, which catalyst comprises a mixed metal oxide, which mixed metal oxide comprises copper, zinc and gallium, wherein the atomic percentage of copper relative to the total number of metal atoms in the oxide is from 20 at. % to 55 at. %.

In another aspect, the invention provides a process for producing a catalyst, which catalyst is suitable for use in a process for producing H₂ by steam reforming of methanol, and which catalyst comprises a mixed metal oxide, which mixed metal oxide comprises copper, zinc and gallium, wherein the atomic percentage of copper relative to the total number of metal atoms in the oxide is from 20 at. % to 55 at. %, which process comprises:

(1) a co-precipitation step, comprising contacting: (a) a solution of copper nitrate, zinc nitrate and gallium nitrate, wherein the atomic percentage of copper relative to the total number of metal atoms in said solution is from 20 at. % to 55 at. %, with (b) a metal carbonate, to produce a co-precipitate comprising said copper, zinc and gallium;

(2) a separation step, comprising separating the co-precipitate from solution; and

(3) a calcination step, comprising calcining the co-precipitate by heating the co-precipitate in air.

The process of the invention for producing a catalyst optionally further comprises:

(4) a reduction step, comprising heating the calcined product in the presence of H₂.

In another aspect, the invention provides a catalyst which is obtainable by the process of the invention for producing a catalyst.

In another aspect, the invention provides the use of a catalyst of the invention as defined above in a process for producing H₂ by steam reforming of methanol.

In another aspect, the invention provides a fuel cell system comprising a fuel cell and a methanol reformer, which methanol reformer comprises a catalyst of the invention. Typically, the fuel cell is a proton exchange membrane (PEM) fuel cell.

In another aspect, the invention provides a portable electronic device comprising a fuel cell system of the invention.

In another aspect, the invention provides the use of a catalyst in a process for producing methanol by the hydrogenation of carbon dioxide, which catalyst comprises a mixed metal oxide, which mixed metal oxide comprises copper, zinc and gallium, wherein the atomic percentage of copper relative to the total number of metal atoms in the oxide is from 20 at. % to 55 at. %.

In another aspect, the invention provides a process for producing methanol by the hydrogenation of carbon dioxide, which process comprises contacting a gas phase comprising (a) CO₂ and (b) H₂, with a solid catalyst, which solid catalyst comprises a mixed metal oxide, which mixed metal oxide comprises copper, zinc and gallium, wherein the atomic percentage of copper relative to the total number of metal atoms in the oxide is from 20 at. % to 55 at. %.

### BRIEF DESCRIPTION OF THE FIGURES

FIG. 1 shows a plot of thermodynamic equilibrium product compositions as function of temperature (calculations performed with the HSC Chemistry® 5.11 software).

FIG. 2 shows a plot of typical catalysts, their methanol conversion & CO content for the steam reforming reaction. (Reaction conditions: 0.40 g cat.+0.40 g SiC; liquid feed of CH₃OH:H₂O=1:2 @ 0.1 mL/min.; N₂ carrier @ 10 mL/min.; 195° C.) A: 43% Cu—ZnGaOx, B: 43% Cu—CeZrOx, C: 43% Cu—ZnAlOx, D: 43% Cu—LaMnOx, E: 43% Cu—ZnOx, F: 43% Cu—CeAlOx, G: 43% Cu—CeGaOx, H: 43% Cu—CeOx, I: 43% Cu—ZrOx, 43% Cu—AlGaOx, K: 43% Cu—ZrGaOx, L: 43% Cu—FeOx, M: 43% Cu—GaOx, N: 43% Cu—ZnCeOx, O: 43% Cu—ZnZrOx, P: 43% Cu—AlOx, Q: 43% Cu—GaZnAlOx, R: 43% Cu—GaCeAlOx, S: 43% Cu—CeZnAlOx, T: HiFUEL R120-JM commercial catalyst.

FIG. 3 shows a plot of catalytic performance, gas content & CO selectivity (and concentration), against reaction temperature for steam reforming of methanol by (a) GoZnGaOₓ; (b) thermodynamic calculations (Reaction conditions: 0.40 g cat.+0.40 g SiC; liquid feed of CH₃OH:H₂O=1:2 @ 0.01 mL/min.; N₂ carrier @ 10 mL/min.; varying temperature).

FIG. 4 shows a plot of catalytic performance, gas content & CO selectivity, against liquid feed rate for the direct steam reforming of methanol by (a) CuZnGaOₓ catalyst; (b) thermodynamic calculations (Reaction conditions: 0.40 g cat.+0.40 g SiC; 195° C.; CH₃OH:H₂O=1:2; N₂ carrier @ 10 mL/min.; varying liquid feed rate).

FIG. 5 shows a plot of catalytic performance, gas content & CO selectivity, against methanol:water molar ratio by (a) CuZnGaOₓ catalyst; (b) thermodynamic calculations (Reaction conditions: 0.40 g cat.+0.40 g SiC; 195° C.; CH₃OH:H₂O=1:2; N₂ carrier @ 10 mL/min.; varying liquid feed rate).

FIG. 6 shows (a) a plot of catalytic performance, gas content & CO selectivity, against contact time with CuZnGaOₓ catalyst. (Reaction conditions: 0.40 g cat.+0.40 g SiC; 150° C.; CH₃OH:H₂O=1:2; N₂ carrier @ 10 mL/min.; varying liquid feed rate); (b) a plot of the hydrogen productivity obtained with the reaction condition as presented in FIG. 6(a).

FIG. 7 shows (a) TPR analysis of the calcined copper samples; (b) TPR analysis of the samples after the TPR treatment, cooled to room temperature prior to further treatment of N₂O.

FIG. 8 shows XRD patterns for as-synthesized (dried at 80° C.) metal oxide samples before calcination with diffraction bands from gallium oxide hydrate (♦) and zinc carbonate hydroxide (▾) identified. The co-precipitated ZnGaOx sample exhibits sharp diffraction bands () for a hydrotalcite-like structured mix oxide. Excess Cu content resulted in the formation of copper carbonate hydrate with its corresponding diffraction bands observed as indicated ( [symbol] ).

FIG. 9 shows XRD patterns for as-synthesized (dried at 80° C.) metal oxide samples before calcination. Without excess Cu content, the LDH-40-CuZnOx sample was able to maintain the hydrotalcite-like structure as compared to the ZnGaOx.

FIG. 10 is a schematic illustration of the reactor setup used in Example 2.

FIG. 11 shows an activity scatter plot for the catalysts synthesised in Example 2.

FIG. 12 shows TPR profiles for calcined ZnO, Ga₂O₃ and ZnGaOₓ.

FIG. 13 shows TPR profiles for 43CuZnOₓ, 43CuGaOₓ, 43CuZnGaOₓ and Cu^{(II)}O.

FIG. 14 shows TPR profiles for catalysts varying the Zn/Ga ratio.

FIG. 15 shows TPR profiles for 43CuZnGaOₓ bulk Cu and surface Cu⁺.

FIG. 16 shows the N₂O chemisorption profile for 43CuZnOₓ.

FIG. 17 shows N₂O chemisorption TPR profiles for 43CuGaOₓ.

FIG. 18 shows N₂O chemisorption TPR profiles for 43CuZnGaOₓ.

FIG. 19 shows N₂O chemisorption TPR profiles for 15CuZnGaOₓ.

FIG. 20 shows N₂O chemisorption TPR profiles for 60CuZnGaOₓ.

FIG. 21 shows N₂O chemisorption TPR profiles for 43CuZn3Ga2Oₓ.

FIG. 22 shows N₂O chemisorption TPR profiles for 43CuZn1Ga3Oₓ.

FIG. 23 shows X-ray diffraction peaks for ZnO before & after calcination at 380° C.

FIG. 24 shows the X-ray diffraction peaks for 43CuZnOₓ.

FIG. 25 shows a comparison between the X-ray diffraction peaks for calcined ZnO and 43CuZnOₓ.

FIG. 26 shows the X-ray diffraction peaks for Ga₂O₃.

FIG. 27 shows the X-ray diffraction peaks for 43CuGaOₓ.

FIG. 28 shows a comparison between the X-ray diffraction peaks for calcined Ga₂O₃ and 43CuGaOₓ.

FIG. 29 shows the X-ray diffraction peaks for ZnGaOₓ.

FIG. 30 shows XRD profiles of calcined ZnO, Ga₂O₃ and ZnGaOₓ.

FIG. 31 shows the XRD profile for 43CuZnGaOₓ.

FIG. 32 shows the XRD profiles of calcined ZnGaOₓ and 43CuZnGaOₓ.

FIG. 33 shows the XRD profiles for calcined 43CuGaOₓ, 43CuZnOₓ, 43CuZnGaOₓ and Cu^{(II)}O.

FIG. 34 shows EPR spectra for 43% CuZnOₓ, CuZnGaOₓ and CuGaOₓ.

FIG. 35 shows a contour map showing variation of SA_{cat} (indicated by colour gradient—or shading gradient in the black and white reproduction; units=m² g⁻¹ cat.) with atomic content.

FIG. 36 is a schematic illustration of a cubic spinel crystal structure.

FIG. 37 shows a contour map showing variation of MeOH conversion (%) with atomic content.

FIG. 38 shows a contour plot showing SA_{cat} affects MeOH conversion & CO production.

FIG. 39 shows a contour map showing how CO production (ppm) varies with atomic content.

FIG. 40 is a schematic illustration of a possible mechanism for the SRM reaction.

FIG. 41 is a schematic illustration of a possible mechanism for the reverse WGS reaction.

### DETAILED DESCRIPTION OF THE INVENTION

The invention provides a process for producing H₂ (hydrogen) by steam reforming of methanol, as defined above. In the process of the invention, the H₂ is advantageously produced directly, in a single step, by Non-Syngas Direct Steam Reforming (NSGDSR) of methanol in accordance with the following reaction:

CH₃OH+H₂O→CO₂+3H₂

This in clear contrast with the conventional complex route involving steam reformation to syngas, followed by water gas shift and CO cleanup stages for the hydrogen production.

The process comprises contacting a gas phase comprising (a) CH₃OH and (b) H₂O, with a solid catalyst. The gas phase may or may not comprise other gases, in addition to the methanol and steam. For instance, the gas phase may comprise an inert gas, e.g. nitrogen or argon, which could for example be present as a carrier gas. The inert gas, when present, is typically nitrogen. Additionally or alternatively, the gas phase may further comprise oxygen. Blending oxygen or air into the gas phase may encourage combustion and may also balance the total thermodynamic requirements of the NSGDSR system. Thus, the gas phase may further comprise oxygen or air.

The gases in the gas phase may be pre-mixed, i.e. mixed together before the mixture is brought into contact with the catalyst. Alternatively, the gases can be fed into a reactor separately, so that the reactant gases are mixed together in the presence of the solid catalyst.

Typically, the step of contacting said gas phase with said solid catalyst comprises passing said gas phase through a reactor comprising said catalyst.

In the process of the invention, H₂ is usually produced in the gaseous state. Thus, the process of the invention for producing H₂ is typically a process for producing hydrogen gas.

The solid catalyst used in the process of the invention for producing H₂ comprises a mixed metal oxide, which mixed metal oxide comprises copper, zinc and gallium. The mixed metal oxide may comprise a plurality of oxide phases. Thus, for instance, in one preferred embodiment, the mixed metal oxide includes a non-stoichiometric cubic spinel phase comprising excess interstitial Cu⁺ ions that are highly dispersed and highly reducible, leading to an enhanced surface area of Cu in the catalyst. The mixed metal oxide may comprise other metals, in addition to copper, zinc and gallium, for instance zirconium. However, in some embodiments, the catalyst does not contain any zirconium. In other embodiments, the mixed metal oxide contains little or no zirconium. Thus, the mixed metal oxide may in some embodiments contain less than or equal to 2.0 at. % Zr, relative to the total number of metal atoms in the mixed metal oxide. Alternatively, copper, zinc and gallium may be the only metals in the mixed metal oxide. As will be understood by the skilled person, much of the copper, zinc and gallium in the mixed metal oxide will be present as cations in the oxide structure. However, the mixed metal oxide may additionally comprise particles of these metals in the oxidation state zero, such as for instance particles of copper metal, Cu⁰. Such Cu⁰ particles may be present on the surface of the mixed metal oxide. The Cu⁰ particles will typically have a mean particle size of less than or equal to 10 nm, or for instance less than or equal to 5 nm.

The atomic percentage of copper in the mixed metal oxide (and “copper” here refers to all copper, including copper cations as well as any Cu⁰ present) relative to the total number of metal atoms in the mixed metal oxide (and “metal atoms” here means all metal atoms, including metal ions and any metal present in oxidation state zero) is from 20 at. % to 55 at. %. The inventors have demonstrated good MeOH conversion levels for such catalysts, and little or no CO production at the ppm level. The inventors have also observed a close correlation between MeOH conversion, CO suppression, and the specific surface area of Cu in the oxide. It has been found that deviations from the range of 20 at. % to 55 at. % copper in either direction leads to a reduced specific surface area of Cu, and reduced MeOH conversion. The inventors expected at first that increasing the Cu content would lead to an increase specific surface area of Cu. However, it is a finding of the present invention that beyond ˜50% Cu loading, the surface area starts to decrease again, and so does the MeOH conversion in the steam reformation of methanol. Also, the inventors have observed that in general, CO production decreases as MeOH conversion increases, which is in turn dependent on the surface area of Cu in the catalyst. Thus, the catalysts of the invention, which are used in the process of the present invention for producing H₂, contain an advantageous level of Cu of from 20 at. % to 55 at. %, which provides for a high surface area, good MeOH conversion and suppression of CO production.

Typically, the atomic percentage of copper in the mixed metal oxide, relative to the total number of metal atoms in the oxide the atomic percentage of copper is from 30 at. % to 55 at. %. More typically, it is from 35 at. % to 55 at. %. The inventors have found that such catalysts have particularly high specific surface areas of Cu and particularly good MeOH conversion levels and suppression of CO. The atomic percentage of copper may for instance be from 40 at. % to 52 at %.

Typically, the atomic percentage of gallium relative to the total number of metal atoms in the oxide is equal to or greater than 5 at. %. The inventors have also found that including gallium in the catalyst leads to suppression of CO production and that increasing the level of gallium leads to particularly low CO levels. In particular, the inventors have found that CO production is not only related to SA_{cat}, but also strongly related to Ga content, with CO levels decreasing as the Ga content is increased. This is an important discovery, as it means that that Ga in the catalyst is playing an active role in the catalytic process by somehow suppressing CO formation. The inventors have observed from AC impedance that Ga in the support leads to decreasing conductivity and increasing thermal activation energy. This is because as the Ga content increases, the poorly crystalline tetragonal NSS phase begins to predominate, which has reduced Cu mobility due to its lack of ordered crystallinity. The oxygen mobility will also be reduced for the same reason. O-vacancies are believed to play a key role in CO formation; therefore the lack of available O-vacancies in the support due to the poorly crystalline nature of the tetragonal NSS phase, caused by the abundance of Ga, would explain the downward trend in CO formation as Ga content is increased. There is, therefore a balance to be struck between maximising the αCu surface area in order to promote MeOH conversion, and maximising the Ga content so as to suppress CO formation. The presence of gallium is also thought to stabilise a highly dispersed, reducible form of Cu.

Thus, preferably, the atomic percentage of gallium relative to the total number of metal atoms in the oxide is equal to or greater than 10 at. %. The atomic percentage of gallium relative to the total number of metal atoms in the oxide may for instance be equal to or greater than 15 at. %. In another embodiment, the atomic percentage of gallium relative to the total number of metal atoms in the oxide is equal to or greater than 18 at. %. In some embodiments, it is equal to or greater than 20 at. %.

Typically, the atomic percentage of gallium relative to the total number of metal atoms in the oxide is from 10 at. % to 40 at. %. The atomic percentage of gallium relative to the total number of metal atoms in the oxide may for instance be from 10 at. % to 35 at. %, or for instance from 15 at % to 35 at %.

The zinc present in the mixed metal oxide is also thought to stabilise a highly dispersed, reducible form of Cu. Thus, the atomic percentage of zinc relative to the total number of metal atoms in the oxide is typically from 10 at. % to 50 at. %. More typically, the atomic percentage of zinc relative to the total number of metal atoms in the oxide is from 15 at. % to 45 at. %.

Particularly high activities and suppression of CO production have been found in catalyst wherein the atomic percentages of copper, zinc and gallium relative to the total number of metal atoms in the mixed metal oxide are from 30 to 55 at. % copper, at least 15 at. % zinc, and at least 10 at. % gallium. Preferably, the atomic percentages of copper, zinc and gallium relative to the total number of metal atoms in the oxide are from 40 to 55 at. % copper, at least 20 at. % zinc, and at least 15 at. % gallium. In some embodiments, the atomic percentages of copper, zinc and gallium relative to the total number of metal atoms in the oxide may be from 40 to 52 at. % copper, at least 25 at. % zinc, and at least 15 at. % gallium.

Preferably, the atomic percentages of copper, zinc and gallium relative to the total number of metal atoms in the oxide are from 30 to 55 at. % copper, from 15 to 45 at. % zinc, and from 10 to 40 at. % gallium. The atomic percentages of copper, zinc and gallium relative to the total number of metal atoms in the oxide may for instance be from 40 to 55 at. % copper, from 20 to 45 at. % zinc, and from 15 to 35 at. % gallium.

In some embodiments of the process of the invention for producing H₂, the atomic percentages of copper, zinc and gallium relative to the total number of metal atoms in the mixed metal oxide are from 40 to 52 at. % copper, from 20 to 40 at. % zinc, and from 15 to 30 at. % gallium, preferably wherein said atomic percentages are from 47 to 52 at. % copper, from 27 to 33 at. % zinc, and from 17 to 23 at. % gallium, more preferably wherein said atomic percentages are about 49 at. % copper, about 31 at. % zinc, and about 20 at. % gallium.

In some embodiments, the metal atoms in the mixed metal oxide used in the process of the invention for producing H₂ consist essentially of said copper, zinc and gallium.

In some embodiments, the metal atoms in the mixed metal oxide used in the process of the invention for producing H₂ consist of said copper, zinc and gallium, i.e. said copper, zinc and gallium may in some embodiments be the only metals in the mixed metal oxide. In one embodiment, the metal atoms in the mixed metal oxide consist of from 20 to 55 at. % copper, from 5 to 40 at. % zinc, and from 5 to 40 at. % gallium. In another embodiment, the metal atoms in the mixed metal oxide consist of x at. % copper, y at. % gallium, and z at. % zinc, wherein x is from 30 to 55, y is from 10 to 40, and z is 100−(x+y). The metal atoms in the mixed metal oxide may for instance consist of x at. % copper, y at. % gallium, and z at. % zinc, wherein x is from 40 to 55, y is from 15 to 35, and z is 100−(x+y). In one preferred embodiment, x is from 40 to 52, y is from 15 to 35, and z is 100−(x+y). In another preferred embodiment, x is from 40 to 55, y is from 15 to 25, and z is 100−(x+y).

Typically, in the process of the invention for producing H₂ (hydrogen) by steam reforming of methanol, the mixed metal oxide has a specific copper metal surface area, as measured by N₂O chemisorption, of at least 40 m²/g-catalyst. The mixed metal oxide may for instance have a specific copper metal surface area, as measured by N₂O chemisorption, of at least 50 m²/g-catalyst, or for instance at least 60 m²/g-catalyst. More preferably, the mixed metal oxide has a specific copper metal surface area, as measured by N₂O chemisorption, of at least 65 m²/g catalyst. In a particularly preferred embodiment the mixed metal oxide has a specific copper metal surface area, as measured by N₂O chemisorption, of at least 70 m²/g catalyst.

Typically, the mixed metal oxide has a specific copper metal surface area per gram copper, as measured by N₂O chemisorption, of at least 100 m²/g-Cu, more typically at least 120 m²/g-Cu or for instance at least 140 m²/g-Cu. Preferably, the surface area per gram copper, as measured by N₂O chemisorption, is at least 150 m²/g-Cu. In some embodiments, it is at least 160 m²/g-Cu, or for instance at least 180 m²/g-Cu. In particularly preferred embodiments the mixed metal oxide has a specific copper metal surface area per gram copper, as measured by N₂O chemisorption, of at least 200 m²/g-Cu. The specific copper metal surface area per gram copper may for instance be at least 210 m²/g-Cu.

Typically, in the process of the invention, the mixed metal oxide comprises particles of said copper, wherein the copper particles have a mean particle size of less than or equal to 40 nm. The particles usually however have a mean particle size of less than or equal to 30 nm, or more typically less than or equal to 20 nm. The term “particle size” as used herein means the diameter of the particle if the particle is spherical or, if the particle is non-spherical, the volume-based particle size. The volume-based particle size is the diameter of the sphere that has the same volume as the non-spherical particle in question.

The mixed metal oxide may for instance comprise particles of said copper, wherein the copper particles have a mean particle size of less than or equal to 10 nm. Preferably, the copper particles have a mean particle size of less than or equal to 5 nm.

Typically, in the process of the invention, the mixed metal oxide has a copper metal dispersion of at least 15%, preferably at least 20%.

Typically, in the process of the invention, the mixed metal oxide comprises Cu²⁺, interstitial Cu⁺ and Cu⁰.

Usually, the mixed metal oxide comprises a non-stoichiometric spinel phase comprising copper, zinc and gallium.

Typically, the mixed metal oxide comprises a non-stoichiometric cubic spinel phase comprising copper, zinc and gallium.

Usually, the spinel phase comprises interstitial Cu⁺. The interstitial Cu⁺ ions are easily reducible to Cu metal (Cu⁰) leading to high Cu⁰ dispersion and surface area.

Thus, the mixed metal oxide typically comprises a spinel phase comprising copper, zinc and gallium, which spinel phase comprises interstitial Cu⁺ and Cu⁰.

The spinel phase typically also comprises octahedral Cu²⁺.

Thus, the mixed metal oxide typically comprises a spinel phase comprising copper, zinc and gallium, which comprises octahedral Cu²⁺, interstitial Cu⁺ and Cu⁰.

The spinel phase typically comprises particles of copper metal (Cu⁰). These copper particles typically have a mean particle size of less than or equal to 40 nm, or for instance less than or equal to 30 nm, or preferably less than or equal to 20 nm. In one preferred embodiment the spinel phase comprises particles of copper metal (Cu⁰) which have a mean particle size of less than or equal to 10 nm. More preferably the copper particles have a mean particle size of less than or equal to 5 nm.

The catalyst used in the process of the invention for producing H₂ may or may not further comprise a solid support material, in addition to said mixed metal oxide. Any suitable support material may be used. In other embodiments, the catalyst does not further comprise a solid support material. Thus, the mixed metal oxide may be unsupported.

The catalyst used in the process of the invention for producing H₂ may be a catalyst which is obtainable by the process of the invention as defined herein for producing a catalyst suitable for use in a process for producing H₂ by steam reforming of methanol.

Usually, in the process of the invention for producing H₂ by steam reforming of methanol, the step of contacting the gas phase with the solid catalyst is performed at atmospheric pressure. However, pressures other than atmospheric pressure may also be used.

The inventors have shown that, in the process of the invention for producing H₂ by steam reforming of methanol, the catalyst can advantageously be used to generate hydrogen, with little or no production of CO, at temperatures of less than or equal to 200° C. However, higher temperatures may in principle be used. Typically, therefore, the step of contacting the gas phase with the solid catalyst is performed at a temperature which does not exceed 200° C.

Usually, the step of contacting the gas phase with the solid catalyst is performed at a temperature of from 80° C. to 200° C., more typically from 100° C. to 200° C.

In some embodiments, the step of contacting the gas phase with the solid catalyst is performed at a temperature of from 120° C. to 200° C., more typically from 130° C. to 200° C. For instance, the step of contacting the gas phase with the solid catalyst may be performed at a temperature of from 140° C. to 200° C.

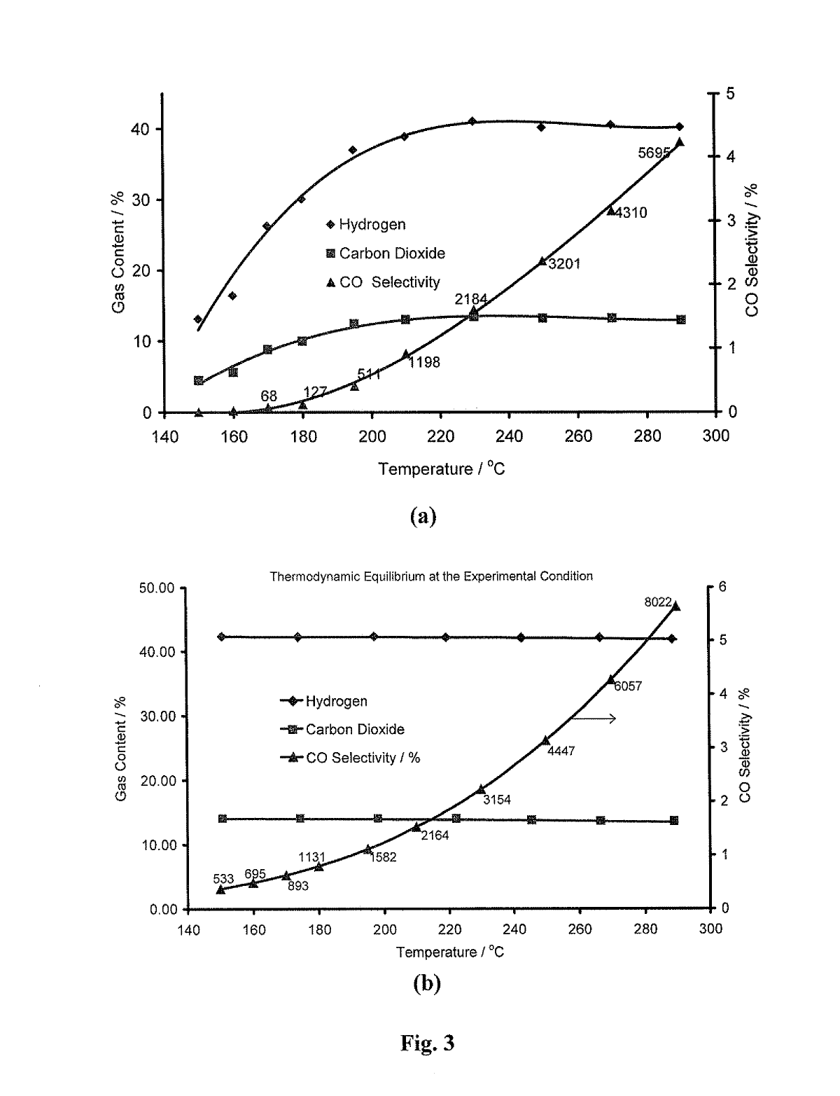

Example 1 herein also shows however that CO formation can be suppressed totally or reduced by decreasing reaction temperature, in order to discourage the slow RWGS reaction; FIG. 3(a) herein shows that there was no CO formation detectable at the ppm level, at or below 150° C. Thus, the step of contacting the gas phase with the solid catalyst may be performed at a temperature which does not exceed 175° C. The step of contacting the gas phase with the solid catalyst may for instance be performed at a temperature of from 100° C. to 175° C. Preferably, however, the step of contacting the gas phase with the solid catalyst is performed at a temperature which does not exceed 150° C. The step of contacting the gas phase with the solid catalyst may for instance be performed at a temperature of from 80° C. to 150° C., or from 100° C. to 150° C., or for instance from 120° C. to 150° C.

The process of the invention for producing H₂ preferably occurs substantially without any formation of carbon monoxide. Thus, the process of the invention typically occurs substantially without any formation of carbon monoxide either via methanol decomposition, as follows:

CH₃OH→CO+2H₂

or via the reverse water-gas shift reaction as follows:

CO₂+H₂→CO+H₂O

Typically, the gaseous product mixture comprises no more than 100 ppm by volume of carbon monoxide, more typically no more than 50 ppm by volume. In preferred embodiments, the gaseous product mixture comprises no more than 10 ppm by volume of carbon monoxide.

Thus, usually, in the process of the invention for producing H₂ by steam reforming of methanol, the process occurs substantially without any formation of carbon monoxide. Typically, the level of CO produced does not exceed 100 ppm. More typically, the level of CO produced does not exceed 50 ppm. In particularly preferred embodiments, the level of CO produced does not exceed 10 ppm.

Typically, in the process of the invention for producing H₂, the percent conversion of methanol is at least 20%. More typically, it is at least 30%.

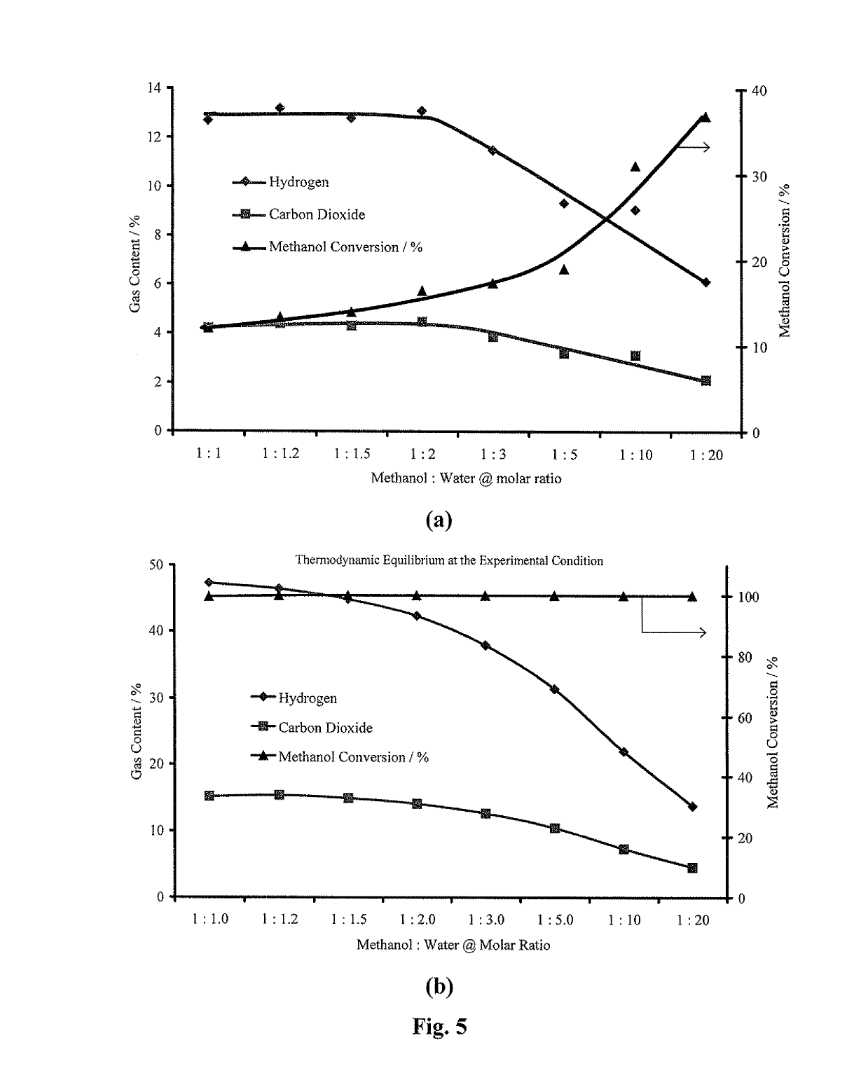

Usually, in the process of the invention for producing H₂, the molar ratio of H₂O to CH₃OH in said gas phase is equal to or greater than 1. In some embodiments however the molar ratio of H₂O to CH₃OH in said gas phase is equal to or greater than 10:1, preferably equal to or greater than 20:1. Such molar ratios were found to promote the methanol conversion; FIG. 5(a) shows that methanol conversion can reach 36%, giving 3:1 H₂/CO₂ with the methanol:water molar ratio set at 1:20. Moreover, it was exciting to note that there was no CO detected for all methanol to water molar ratios at 150° C. Thus, preferably, the step of contacting the gas phase with the solid catalyst is performed at a temperature which does not exceed 150° C. and using a molar ratio of H₂O to CH₃OH in said gas phase which is equal to or greater than 1:1, preferably equal to or greater than 3:1, more preferably equal to or greater than 10:1.

In the process of the invention for producing H₂, the gas phase which comprises H₂O and CH₃OH may be generated by feeding a liquid phase comprising said H₂O and CH₃OH through a heated zone, which causes evaporation of the liquid phase to produce said gas phase. Typically, in such embodiments, the liquid phase is fed into said heated zone at a feed rate which is equal to or greater than 0.01 mL/minute. However, it is clear from the experiments in Example 1 herein that CO formation can be suppressed or severely reduced by decreasing contact time in order to discourage the slow RWGS reaction that produces CO. Thus, preferably, the liquid phase is fed into said heated zone at a feed rate which is equal to or greater than 0.04 mL/minute, more preferably at a feed rate which is equal to or greater than 0.06 mL/minute, or for instance at a feed rate which is equal to or greater than 0.08 mL/minute. In some embodiments, the liquid phase is fed into said heated zone at a feed rate which is equal to or greater than 0.1 mL/minute, for instance equal to or greater than 0.12 mL/minute.

The process of the invention for producing H₂ by steam reforming of methanol may further comprise recovering said H₂. Typically, the process of the invention produces a mixture of gases comprising H₂ and CO₂. The step of recovering said H₂ typically therefore comprises collecting the product gas mixture and separating the H₂ from said mixture. The separation may be effected by any suitable method known in the art, for instance by using a filter material which selectively retains contaminants and lets the hydrogen pass through. The separated H₂ gas may also for instance be compressed and/or stored for later use.

The process of the invention for producing H₂ by steam reforming of methanol may further comprise using the H₂ produced as a fuel. For instance, the H₂ produced may be used to power a fuel cell, such as a PEM fuel cell.

The catalysts used in the process of the invention for producing H₂ are themselves novel. Accordingly, the invention further provides a catalyst for use in a process for producing H₂ by steam reforming of methanol, which catalyst comprises a mixed metal oxide, which mixed metal oxide comprises copper, zinc and gallium, wherein the atomic percentage of copper relative to the total number of metal atoms in the oxide is from 20 at. % to 55 at. %.

The catalyst of the invention may be as further defined hereinbefore, in the discussion of the process of the invention for producing H₂ by steam reforming of methanol. Thus, the catalyst of the invention comprises a mixed metal oxide, which mixed metal oxide comprises copper, zinc and gallium. The mixed metal oxide may comprise a plurality of oxide phases. Thus, for instance, in one preferred embodiment, the mixed metal oxide in the catalyst of the invention includes a non-stoichiometric cubic spinel phase comprising excess interstitial Cu⁺ ions that are highly dispersed and highly reducible, leading to an enhanced surface area of Cu in the catalyst. The mixed metal oxide may comprise other metals, in addition to copper, zinc and gallium, for instance zirconium. In other embodiments, the mixed metal oxide contains little or no zirconium. Thus, the mixed metal oxide may in some embodiments contain less than or equal to 2.0 at. % Zr, relative to the total number of metal atoms in the mixed metal oxide. In one embodiment, the catalyst does not contain any zirconium. Alternatively, copper, zinc and gallium may be the only metals in the mixed metal oxide. As will be understood by the skilled person, much of the copper, zinc and gallium in the mixed metal oxide will be present as cations in the oxide structure. However, the mixed metal oxide may additionally comprise particles of these metals in the oxidation state zero, such as for instance particles of copper metal, Cu⁰. Such Cu⁰ particles may be present on the surface of the mixed metal oxide. The Cu⁰ particles will typically have a mean particle size of less than or equal to 10 nm, or for instance less than or equal to 5 nm.

The atomic percentage of copper in the mixed metal oxide of the catalyst of the invention, relative to the total number of metal atoms in the mixed metal oxide, is from 20 at. % to 55 at. %. More typically, the atomic percentage of copper in the mixed metal oxide, relative to the total number of metal atoms in the oxide the atomic percentage of copper is from 30 at. % to 55 at. %. Even more typically, it is from 35 at. % to 55 at. %. The atomic percentage of copper may for instance be from 40 at. % to 52 at %.

Typically, the atomic percentage of gallium relative to the total number of metal atoms in the mixed metal oxide of the catalyst of the invention is equal to or greater than 5 at. %. Preferably, the atomic percentage of gallium relative to the total number of metal atoms in the oxide is equal to or greater than 10 at. %. The atomic percentage of gallium relative to the total number of metal atoms in the oxide may for instance be equal to or greater than 15 at. %. In another embodiment, the atomic percentage of gallium relative to the total number of metal atoms in the oxide is equal to or greater than 18 at. %. In some embodiments, it is equal to or greater than 20 at. %.

Typically, the atomic percentage of gallium relative to the total number of metal atoms in the oxide is from 10 at. % to 40 at. %. The atomic percentage of gallium relative to the total number of metal atoms in the oxide may for instance be from 10 at. % to 35 at. %, or for instance from 15 at % to 35 at %.

The atomic percentage of zinc relative to the total number of metal atoms in the oxide is typically from 10 at. % to 50 at. %. More typically, the atomic percentage of zinc relative to the total number of metal atoms in the oxide is from 15 at. % to 45 at. %.

In some embodiments of the catalyst of the invention, the atomic percentages of copper, zinc and gallium relative to the total number of metal atoms in the mixed metal oxide are from 30 to 55 at. % copper, at least 15 at. % zinc, and at least 10 at. % gallium. Preferably, the atomic percentages of copper, zinc and gallium relative to the total number of metal atoms in the oxide are from 40 to 55 at. % copper, at least 20 at. % zinc, and at least 15 at. % gallium. In some embodiments, the atomic percentages of copper, zinc and gallium relative to the total number of metal atoms in the oxide may be from 40 to 52 at. % copper, at least 25 at. % zinc, and at least 15 at. % gallium.

More preferably, the atomic percentages of copper, zinc and gallium relative to the total number of metal atoms in the oxide are from 30 to 55 at. % copper, from 15 to 45 at. % zinc, and from 10 to 40 at. % gallium. The atomic percentages of copper, zinc and gallium relative to the total number of metal atoms in the oxide may for instance be from 40 to 55 at. % copper, from 20 to 45 at. % zinc, and from 15 to 35 at. % gallium.

In some embodiments of the catalyst of the invention, the atomic percentages of copper, zinc and gallium relative to the total number of metal atoms in the mixed metal oxide are from 40 to 52 at. % copper, from 20 to 40 at. % zinc, and from 15 to 30 at. % gallium, preferably wherein said atomic percentages are from 47 to 52 at. % copper, from 27 to 33 at. % zinc, and from 17 to 23 at. % gallium, more preferably wherein said atomic percentages are about 49 at. % copper, about 31 at. % zinc, and about 20 at. % gallium.

In some embodiments of the catalyst of the invention, the metal atoms in the mixed metal oxide consist essentially of said copper, zinc and gallium. In some embodiments, the metal atoms in the mixed metal oxide consist of said copper, zinc and gallium, i.e. said copper, zinc and gallium may in some embodiments be the only metals in the mixed metal oxide. Thus, the metal atoms in the mixed metal oxide may consist of from 20 to 55 at. % copper, from 5 to 40 at. % zinc, and from 5 to 40 at. % gallium. In another embodiment, the metal atoms in the mixed metal oxide consist of x at. % copper, y at. % gallium, and z at. % zinc, wherein x is from 30 to 55, y is from 10 to 40, and z is 100−(x+y). The metal atoms in the mixed metal oxide may for instance consist of x at. % copper, y at, % and z at. % zinc, wherein x is from 40 to 55, y is from 15 to 35, and z is 100−(x+y). In one preferred embodiment, x is from 40 to 52, y is from 15 to 35, and z is 100−(x+y). In another preferred embodiment, x is from 40 to 55, y is from 15 to 25, and z is 100−(x+y).

Typically, in the catalyst of the invention, the mixed metal oxide has a specific copper metal surface area, as measured by N₂O chemisorption, of at least 40 m²/g-catalyst. The mixed metal oxide may for instance have a specific copper metal surface area, as measured by N₂O chemisorption, of at least 50 m²/g-catalyst, or for instance at least 60 m² g-catalyst. More preferably, the mixed metal oxide has a specific copper metal surface area, as measured by N₂O chemisorption, of at least 65 m²/g catalyst. In a particularly preferred embodiment the mixed metal oxide has a specific copper metal surface area, as measured by N₂O chemisorption, of at least 70 m²/g catalyst.

Typically, the mixed metal oxide has a specific copper metal surface area per gram copper, as measured by N₂O chemisorption, of at least 100 m²/g-Cu, more typically at least 120 m²/g-Cu or for instance at least 140 m²/g-Cu. Preferably, the surface area per gram copper, as measured by N₂O chemisorption, is at least 150 m²/g-Cu. In some embodiments, it is at least 160 m²/g-Cu, or for instance at least 180 m²/g-Cu. In particularly preferred embodiments the mixed metal oxide has a specific copper metal surface area per gram copper, as measured by N₂O chemisorption, of at least 200 m²/g-Cu. The specific copper metal surface area per gram copper may for instance be at least 210 m²/g-Cu.

Typically, in the catalyst of the invention, the mixed metal oxide comprises particles of said copper, wherein the copper particles have a mean particle size of less than or equal to 40 nm. The particles usually however have a mean particle size of less than or equal to 30 nm, or more typically less than or equal to 20 nm. The term “particle size” as used herein means the diameter of the particle if the particle is spherical or, if the particle is non-spherical, the volume-based particle size. The volume-based particle size is the diameter of the sphere that has the same volume as the non-spherical particle in question.

The mixed metal oxide may for instance comprise particles of said copper, wherein the copper particles have a mean particle size of less than or equal to 10 nm. Preferably, the copper particles have a mean particle size of less than or equal to 5 nm.

Typically, in the catalyst of the invention, the mixed metal oxide has a copper metal dispersion of at least 15%, preferably at least 20%.

Typically, in the catalyst of the invention, the mixed metal oxide comprises Cu²⁺, Cu⁺ and Cu⁰. The Cu⁺ is typically interstitial Cu⁺.

Usually, the mixed metal oxide comprises a non-stoichiometric spinel phase comprising copper, zinc and gallium.

Typically, the mixed metal oxide comprises a non-stoichiometric cubic spinet phase comprising copper, zinc and gallium.

Usually, the spinel phase comprises interstitial Cu⁺. The interstitial Cu⁺ ions are easily reducible to Cu metal (Cu⁰) leading to high Cu⁰ dispersion and surface area.

Thus, the mixed metal oxide typically comprises a spinel phase comprising copper, zinc and gallium, which spinel phase comprises interstitial Cu⁺ and Cu⁰.

The spinel phase typically also comprises octahedral Cu²⁺.

Thus, the mixed metal oxide typically comprises a spinel phase comprising copper, zinc and gallium, which comprises octahedral Cu²⁺, interstitial Cu⁺ and Cu⁰.

The spinel phase typically comprises particles of copper metal (Cu⁰). These copper particles typically have a mean particle size of less than or equal to 40 nm, or for instance less than or equal to 30 nm, or preferably less than or equal to 20 nm. In one preferred embodiment the spinel phase comprises particles of copper metal (Cu⁰) which have a mean particle size of less than or equal to 10 nm. More preferably the copper particles have a mean particle size of less than or equal to 5 nm.

The catalyst of the invention may or may not further comprise a solid support material, in addition to said mixed metal oxide. Any suitable support material may be used. In other embodiments, the catalyst of the invention does not further comprise a solid support material. Thus, the mixed metal oxide may be unsupported.

Typically, the catalyst of the invention is obtainable by the process of the invention defined herein for producing a catalyst, which catalyst suitable for use in a process for producing H₂ by steam reforming of methanol.

Thus, further provided is a process for producing a catalyst, which catalyst is suitable for use in a process for producing H₂ by steam reforming of methanol, and which catalyst comprises a mixed metal oxide, which mixed metal oxide comprises copper, zinc and gallium, wherein the atomic percentage of copper relative to the total number of metal atoms in the oxide is from 20 at. % to 55 at. %, which process comprises: (1) a co-precipitation step, comprising contacting: (a) a solution of copper nitrate, zinc nitrate and gallium nitrate, wherein the atomic percentage of copper relative to the total number of metal atoms in said solution is from 20 at. % to 55 at. %, with (b) a metal carbonate, to produce a co-precipitate comprising said copper, zinc and gallium; (2) a separation step, comprising separating the co-precipitate from solution; and (3) a calcination step, comprising calcining the co-precipitate by heating the co-precipitate in air. The process usually further comprises: (4) a reduction step, comprising heating the calcined product in the presence of H₂.

As the skilled person will appreciate, the proportions of copper, zinc and gallium in the catalyst can be accurately controlled by varying the proportions of copper nitrate, zinc nitrate and gallium nitrate employed in the solution used in step (1). The atomic percentages of copper, zinc and gallium in the final mixed metal oxide product should be the same as the atomic percentages of copper, zinc and gallium in the solution of the respective nitrates. Thus, any of the catalysts of the invention defined above, having any of the abovementioned atomic percentages of copper, zinc and gallium, can be produced by dissolving the correct amounts of copper nitrate, zinc nitrate and gallium nitrate in the solution used in step (1).

Accordingly, in the process of the invention for producing a catalyst, the atomic percentages of copper, zinc and/or gallium in said solution of copper nitrate, zinc nitrate and gallium nitrate, relative to the total number of metal atoms in said solution, may be the same as any of the atomic percentages of copper, zinc and/or gallium defined herein for the catalyst of the invention.

Usually, the co-precipitation step comprises contacting: (a) said solution of said copper nitrate, zinc nitrate and gallium nitrate, which is an aqueous solution, with (b) a second aqueous solution which comprises said metal carbonate. Said contacting may be performed by adding both solutions to water, usually deionised water. Typically, the contacting is performed whilst stirring. Typically, the contacting is carried out at a pH of from 6 to 7.

Any suitable metal carbonate may be used in the process of the invention for producing a catalyst. Typically, the metal carbonate is an alkali metal carbonate, for instance sodium carbonate.

The co-precipitation step may further comprise an aging step. The aging step typically comprises heating the co-precipitate in the presence of a solvent, typically the solvent from which it was precipitated (usually water). Typically, the co-precipitate is heated to a temperature of up to about 80° C., or for instance up to about 90° C. The co-precipitate may be heated at the temperature for up to about 24 hours.

In the separation step, any suitable means can be used to separate the co-precipitate from solution. For instance, the separation may be performed by filtration or by centrifugation. Typically, the separation step further comprises washing the co-precipitate, after separating the co-precipitate from solution. Typically, the co-precipitate is washed with distilled water. The separation step may additionally further comprise drying the co-precipitate. The co-precipitate is typically dried at a temperature of equal to or greater than 70° C., e.g. at a temperature of from 80 to 100° C. It is typically dried at the temperature for a number of hours, e.g. for 4 hours or more. It is typically dried at the temperature for 8 to 16 hours. The co-precipitate is usually dried in air.

The calcination step typically comprises heating the co-precipitate in air to a temperature of at least 250° C. More typically, the co-precipitate is heated in air to a temperature of at least 350° C., or for instance to a temperature of at least 380° C. Typically, in the calcination step, the co-precipitate is heated in air at the temperature for at least 1 hour, more typically for at least 2 hours. A typical heating programme would be ramping at 3° C./min up to 380° C. for 180 mins. The co-precipitate is typically heated to the temperature in static air.

Usually, the reduction step comprises heating the calcined product in the presence of H₂ (typically in the presence of a mixture of H₂ and an inert gas, such as N₂, and more typically under a flowing stream of H₂ and the inert gas). The calcined product may be heated in the presence of said H₂ to a temperature of at least 120° C. More typically, the reduction step comprises heating the calcined product in the presence of said H₂ to a temperature of at least 150° C. The calcined product is typically heated in the presence of said H₂ for up to about 2 hours.

The catalyst produced by the process of the invention may be as further defined herein for the catalyst of the invention.

The process of the invention for producing a catalyst typically further comprises recovering the catalyst.

The process of the invention for producing a catalyst may further comprise using the catalyst thus produced for producing H₂ by steam reforming of methanol. Thus, the process may further comprise using the catalyst thus produced in a process of the invention as defined herein for producing H₂ by steam reforming of methanol.

The invention further provides a catalyst which is obtainable by a process of the invention as defined herein for producing a catalyst.

The invention further provides the use of a catalyst of the invention as defined herein in a process for producing H₂ by steam reforming of methanol.

The invention further provides the use of a catalyst, which catalyst is obtainable by the process of the invention as defined herein for producing a catalyst, in a process for producing H₂ by steam reforming of methanol.

The catalysts of the invention can be used to produce hydrogen, in accordance with the process of the invention for producing H₂ by steam reforming of methanol, and the hydrogen thus produced may be used to power a fuel cell. Thus, the invention further provides a fuel cell system which comprises (a) a fuel cell and (b) a methanol reformer, wherein the methanol reformer comprises a catalyst of the invention as defined herein. The fuel cell is typically a proton exchange membrane (PEM) fuel cell.

Particularly, for portable applications such as cell phones, mp3-players, laptop computers and similar niche products, the use of PEM fuel cells is deemed to be more energy efficient than battery technology. Low temperature PEM fuel cells are potentially the preferred choices for these consumer products. Thus, in another aspect, the invention provides a portable electronic device comprising a fuel cell system of the invention as defined above. The portable electronic device may for instance be a laptop computer, a mobile interne device, a mobile phone, an MP3 player, a remote control device, a netbook, a video recording device, a camera, a portable military device, a satellite navigation device, or a handheld games console.

It is a further finding of the invention that the catalyst of the invention is surprisingly active for the production of methanol by the hydrogenation of carbon dioxide, in accordance with the following reaction:

CO₂+3H₂→CH₃OH+H₂O

As demonstrated in Example 3 hereinbelow, methanol was produced at a higher yield, and with a higher % conversion of CO₂, and with higher % selectivity for methanol, than when a conventional industrial catalyst (Johnson Matthey HiFUELT R120 catalyst) was used under the same conditions.

Accordingly, the invention further provides the use of a catalyst of the invention as defined herein in a process for producing methanol by the hydrogenation of carbon dioxide.

The invention further provides the use of a catalyst, which catalyst is obtainable by the process of the invention as defined herein for producing a catalyst, in a process for producing methanol by the hydrogenation of carbon dioxide.

Further provided is a process for producing methanol by the hydrogenation of carbon dioxide, which process comprises contacting a gas phase comprising (a) CO₂ and (b) H₂, with a solid catalyst, which solid catalyst comprises a mixed metal oxide, which mixed metal oxide comprises copper, zinc and gallium, wherein the atomic percentage of copper relative to the total number of metal atoms in the oxide is from 20 at. % to 55 at. %.

The catalyst used in this process may be a catalyst of the invention as further defined herein.

Typically, in the process of the invention for producing methanol by the hydrogenation of carbon dioxide, said contacting of the gas phase with the solid catalyst is performed at elevated temperature. Said contacting step is typically performed at a temperature equal to or greater than 400K, more typically at a temperature equal to or greater than 450K. In a preferred embodiment, the process comprises contacting the gas phase with the solid catalyst at a temperature equal to or greater than 500K.

Usually, in the process of the invention for producing methanol by the hydrogenation of carbon dioxide, said contacting of the gas phase with the solid catalyst is performed at elevated pressure (i.e. greater than atmospheric pressure). Said contacting is typically performed at a pressure which is equal to or greater than 2 MPa, more typically at a pressure equal to or greater than 3 MPa. In a preferred embodiment, the process comprises contacting the gas phase with the solid catalyst at a pressure which is equal to or greater than 5 MPa.

The molar ratio of H₂ to CO₂ in said gas phase is usually 3:1, although other molar ratios of these reactant gases may be used as appropriate.

The process of the invention for producing methanol by the hydrogenation of carbon dioxide may further comprise recovering said methanol.

The catalyst used in the process of the invention for producing methanol may be a catalyst which is obtainable by a process of the invention as defined herein for producing a catalyst.

Also, the process of the invention as defined herein for producing a catalyst may further comprise: using the catalyst thus produced for producing methanol by the hydrogenation of carbon dioxide. In particular, the process of the invention as defined herein for producing a catalyst may further comprise: using the catalyst thus produced in a process of the invention as defined herein for producing methanol by the hydrogenation of carbon dioxide.

The present invention is further illustrated in the Examples which follow:

### EXAMPLES

### Example 1

### Non-Syngas Direct Steam Reforming (NSGDSR) of Methanol to Hydrogen and Carbon Dioxide Over CuZnGaOx Catalysts at Low Temperature

In this study, NSGDSR has been carried out at atmospheric pressure, temperature range of 150-200° C., steam to methanol molar ratios ranging from 1-20. Effects of reaction temperature, contact-time, steam to methanol molar ratio and catalyst composition on methanol conversion, CO selectivity, and hydrogen productivity are thus evaluated.

### Catalyst Preparation

Typically, Cu based catalysts such as CuZnGaOₓ, were co-precipitated from a 100 mL aqueous solution containing 3.03 g of Cu(NO₃)₂.xH₂O (Aldrich), 2.40 g of Zn(NO₃)₂.6H₂O (Aldrich) and 2.15 g of Ga(NO₃)₃.xH₂O (Aldrich) by using a Na₂CO₃ aqueous solution (prepared by dissolving 3.50 g of Na₂CO₃ in 100 mL DI water), both solutions were dispensed at 0.05 mL/sec to a high-speed stirring (1500 r/min) 300 mL DI water, with the pH controlled between 6 and 7. Then, the resulting precipitate was aged in the solution at 80-90° C. in a closed system for 24 hours. After aging, the precipitate was recovered by centrifugation, washed with 50 mL distilled water for 6-12 times under suction and dried in air (80-100° C. for 8-16 hours) and subsequently calcined in static air (at 3° C./min up to 380° C. for 180 mins) to produce the catalyst. The calcined catalyst was pre-reduced during temperature programmed reduction from room temperature to 150° C. at 20 mL/min flowing stream of 10% H₂/N₂ for 2 hours prior catalyst testing. Table 1 summarizes the quantities of chemical precursors for CuZnGaOₓ and others related materials using the same synthesis method.

TABLE 1
The quantities of metal precursors used in the synthesis of catalysts tested in this study

| | Cu(NO₃)₂•xH₂O/g | Zn(NO₃)₂•6H₂O/g | Ga(NO₃)₃•xH₂O/g |
|---|---|---|---|
| CuZnGaOₓ-I | 3.03 | 2.40 | 2.15 |
| CuZnGaOₓ-II | 0.54 | 2.40 | 2.15 |
| CuZnGaOₓ-III | 3.03 | 1.20 | 1.08 |
| CuZnOₓ | 3.03 | 4.80 | 0 |
| CuGaOₓ | 3.03 | 0 | 4.30 |
| ZnGaOₓ | 0 | 4.80 | 4.30 |
| ZnOₓ | 0 | 2.40 | 0 |
| GaOₓ | 0 | 0 | 2.15 |

| | |
|---|---|
| HiFUEL ™ R120 | Johnson Matthew—Copper based methanol reforming catalyst |

### Characterisation

Determination of catalytic activity was carried out using a home-built plug-flow reactor system. Typically, a powder mixture of 0.40 g catalyst and 0.40 g SiC sandwiched by silica wool plugs was placed in a 4 mm i.d. silica reactor housed in a temperature programmed furnace. A liquid feed of CH₃OH:H₂O=1:2 set at the flow rate of 0.1 mL/min generated from an HPLC pump was mixed with a N₂ flow at 10 mL/min. The mixture was allowed to pass through pre-heated ⅛″ id. piping at 150° C. where all liquids generated to gas mixture before it reached to the reactor. The exited gas after a dry-ice cold trap (to remove water and methanol) was analyzed with pre-calibrated gas chromatography (GC). Thus, the H₂, CO, CO₂, N₂ and CH₄ gases were measured by GC equipped with thermal conductivity detector. A separate GC-FID (flame ionization detector) equipped with methanator with detection limit of CO below 10 ppm was also used.

TPR experiments were carried out to determine Cu surface areas, to estimate the Cu dispersion and to calculate the corresponding Cu particle size of the Cu particle in the catalyst by N₂O passivation method (Hinrichsen, O.; Genger, T.; Muhler, M. Chem. Eng. Technol. 23, 956-959, 2000). This method is based on the measurement of the hydrogen consumption under temperature programmed reduction of sample (pre-calcined at 380° C.) after reduction followed by surface oxidation by N₂O treatment at room temperature. Here, a small quantity of catalyst sample, 26 mg, was used for the measurement to avoid saturating the instrumental detection limit. In order to get an accurate and reproducible Cu surface area, a low temperature (ramped at 10° C./min to 150° C. and kept there for 5 min before cooling to room temperature) pre-treatment of sample under He (10 mL/min) was used. This was followed by the 1^{st}-TPR analysis using a 20 mL/min flow of 5% H₂/Ar with a temperature programmed ramping rate at 10° C./min to 330° C. and dwelled there for 30 minutes before cooled to room temperature. Thus, the hydrogen consumption corresponding to the reduction of the bulk Cu²⁺ to Cu⁰ was measured. Then, the N₂O treatment was performed at room temperature where the sample was flushed with a 20 mL/min of 5% N₂O/Argon for 40 minutes before it was swept with He (10 mL/min) for 10 minutes to remove any un-reacted N₂O. Then, a 2^{nd}-TPR analysis was conducted using a 20 mL/min flow of 5% H₂/Ar with temperature programmed ramping rate at 10° C./min to 330° C. Thus, the hydrogen consumption corresponding to the reduction of Cu⁺ to Cu⁰ was measured. Thus, after the 1^{st}-TPR, the total amount of reducible copper oxide (CuO+H₂→Cu+H₂O) was determined and after the 2^{nd}-TPR, the number of surface Cu atoms (Cu₂O+H₂→2Cu+H₂O) was determined. The Cu metal dispersion was calculated as follows: [(Bulk Cu from 1^{st}-TPR)÷(Surface Cu from 2^{nd}-TPR)]×100%. The specific Cu metal surface area was calculated to be (Surface Cu atom per gram÷Surface packing density of Cu metal). The specific Cu metal surface area per gram Cu was calculated as: (Specific Cu metal surface area)÷(Cu loading). The Cu particle size (assume spherical) was calculated to be: {6÷[(Specific Cu metal surface area per gram Cu)×(Density of Cu metal)]}. It is noted that all the calculations are valid only by taking an assumption that each surface Cu atom decomposes N₂O to N₂ with the formation of Cu₂O.

### Catalyst Testing Over Steam Reforming of Methanol

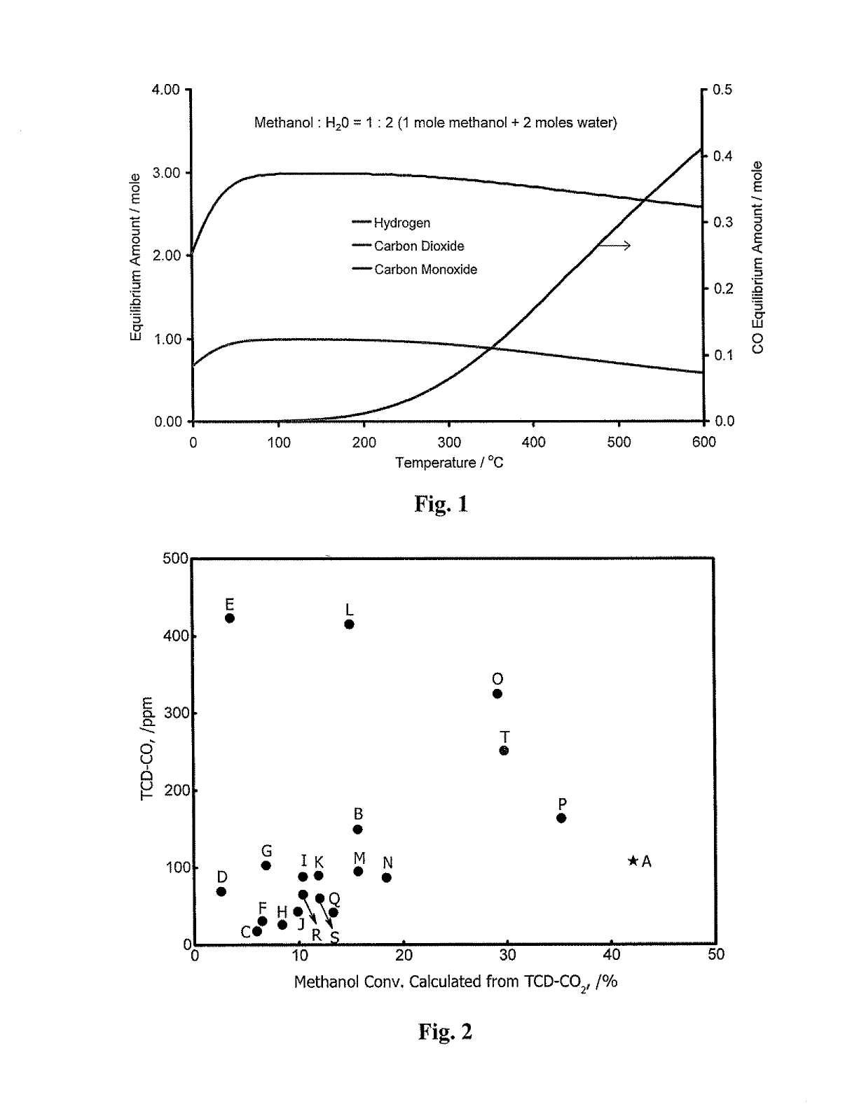

It is important to appreciate the CO content according to equilibrium thermodynamics of the steam reforming of methanol under our reaction conditions. Theoretical calculations were thus performed using commercial software to derive the equilibrium thermodynamics values (HSC Chemistry® 5.11) where only the intrinsic properties of gas species are considered. Here, 1 mole of CH₃OH_{(g)} and 2 mole of H₂O_{(g)} were considered as substrates and product species of CO_{(g)}, CO_{2(g)} & H_{2(g)} were taken into account (experimentally identified). FIG. 1 show clearly that the methanol conversion can reach 100% to carbon dioxide and hydrogen at around 100° C. But, an increase in reaction temperature will favor the CO formation (via reverse water gas shift reaction). The increase in CO content will decrease the production of H₂ and CO₂ from the steam reforming of methanol.

FIG. 2 shows the comparison of the various Cu based catalysts in a fixed bed reactor for the possible direct production of hydrogen and carbon dioxide from steam reformation of methanol. The obtained H₂/CO₂ molar ratios from most catalysts under this condition were of around 3:1 with low CO contents according to the stoichiometry indicative the NSGDSR can be taken place under the reaction conditions. It is however, most catalysts were not active for the direct steam reformation of methanol (<20%). It is interesting that the CuZnGaOx based catalysts give good methanol conversion but low CO content (100 ppm) which are below thermodynamically expected values. Thus, the most active and selective CuZnGaOₓ catalyst was selected for further study.

FIG. 3 shows that the methanol conversion reaches 100%, giving stable 3:1 H₂ to CO₂ at above 230° C. The corresponding CO contents (selectivity) increase at increasing reaction temperature. These values follow well with the thermodynamic calculations (small deviations due to experimental errors in the substrate controls and analyses) at above 230° C. but at below the temperature, it is obviously a kinetic control regime. It is noted that at 150° C. over the CuZnGaOₓ catalyst there was no detectable CO instead of 533 ppm CO predicted from the theoretical calculation.

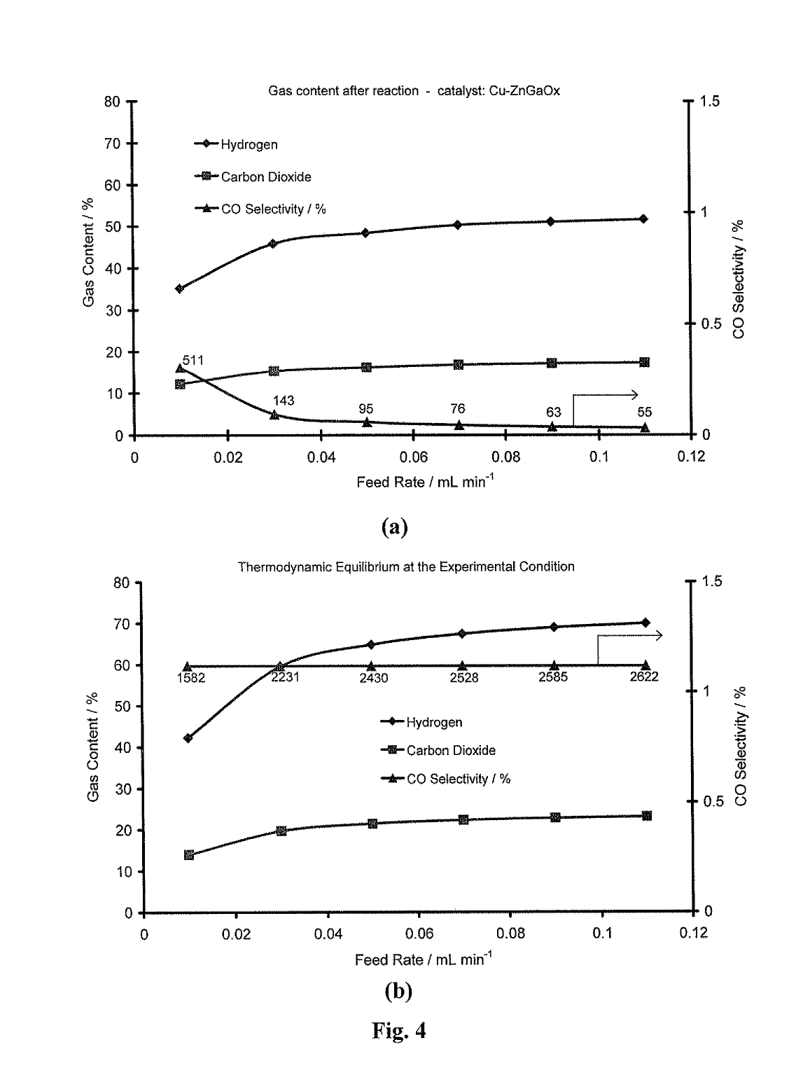

The contact time study of the methanol/water over the catalyst by alternating the liquid feed rate was carried out. As shown in FIG. 4, there was a 511 ppm CO contamination in the product gas at 195° C. at the liquid feed rate of 0.01 mL/min. From there the reaction temperature was kept identical but the methanol-water feeding rate was varied. FIG. 4(a) shows clearly a declining CO content when the liquid feed rate was increased. It is noted that the CO selectivity of the steam reforming of methanol was moving away from its thermodynamic equilibrium value when the catalyst contact time was decreased, as shown in FIG. 4(b). This result indicates that CO₂/H₂ are likely to be the primary products but CO is a secondary product probably via the reversed water gas shift (RWGS) reaction which is a slow reaction under the reaction conditions. The use of short contact time obviously enables the suppression of CO content with respect to equilibrium.

It is thus clear that the key CO formation could be totally suppressed or severely reduced either by decreasing reaction temperature or contact time where the slow RWGS reaction is discouraged. Since there appeared to give no CO formation at or below 150° C., as shown in FIG. 3(a), the next attempt was to promote the methanol conversion while keeping the CO formation at its minimum. Different concentration of methanol in water liquid feed was therefore employed at the total liquid feed of 0.01 mL/min in N₂ at 10 mL/min over the catalyst at 150° C. It is exciting to note that there was no CO detected for all methanol to water ratios at this temperature. FIG. 5(a) shows that methanol conversion can reach 36%, giving 3:1 H₂/CO₂ with the methanol:water molar ratio set at 1:20.

It is useful to appreciate the hydrogen productivity from this low temperature NSGDSR over the CuZnGaOₓ catalyst at 150° C. where no detectable CO is evident. Thus, the hydrogen productivities were evaluated at different methanol-water liquid feeding rates while keeping the other reaction parameters constant. FIG. 6(a) shows that there is a linear relationship between contact time and the methanol conversion at 150° C. Further increase in methanol conversion to give the primary CO₂/H₂ products is expected at longer contact time without producing the CO gas. FIG. 6(b) gives the corresponding hydrogen productivities based on the methanol conversions in FIG. 6(a). It is derived from the FIG. 6(b) that the best hydrogen productivity so far is obtained at 2.624 mL-H₂/min under our testing conditions where the corresponding methanol-water feeding rate was kept at 0.2 mL/min. This corresponds to a hydrogen productivity of 393.6 mL-H₂/g cat./hour.

### Catalyst Characterisation

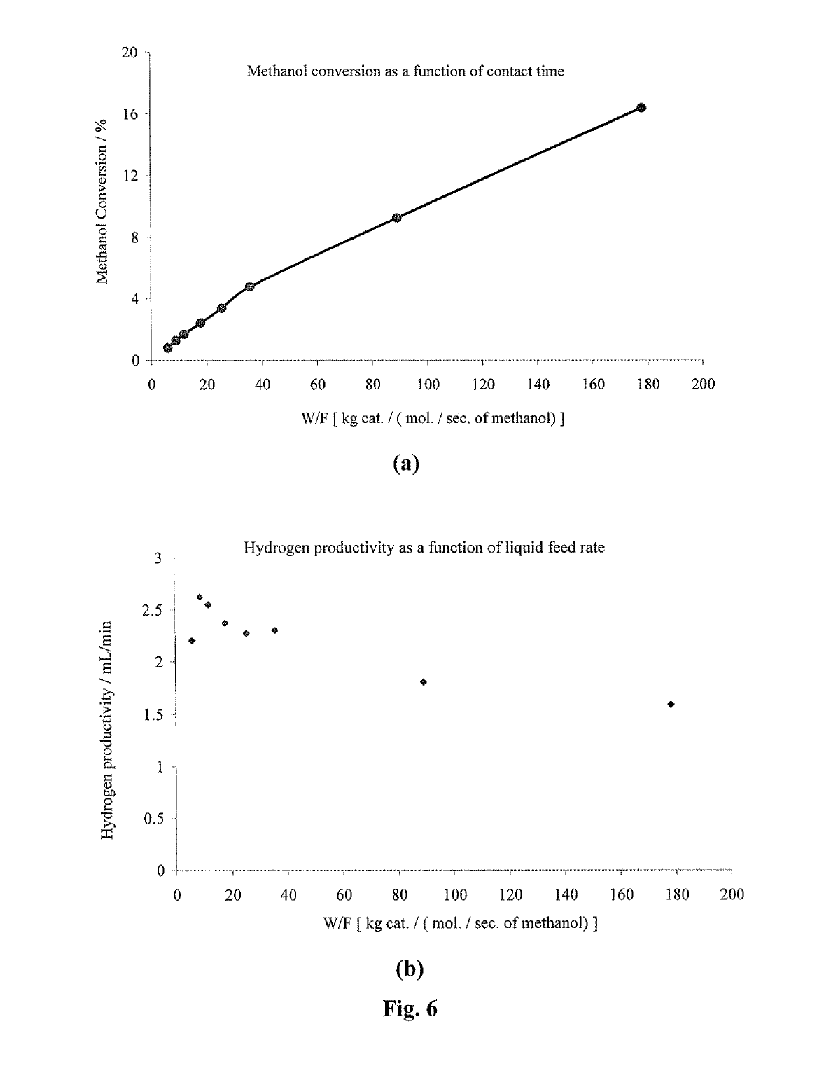

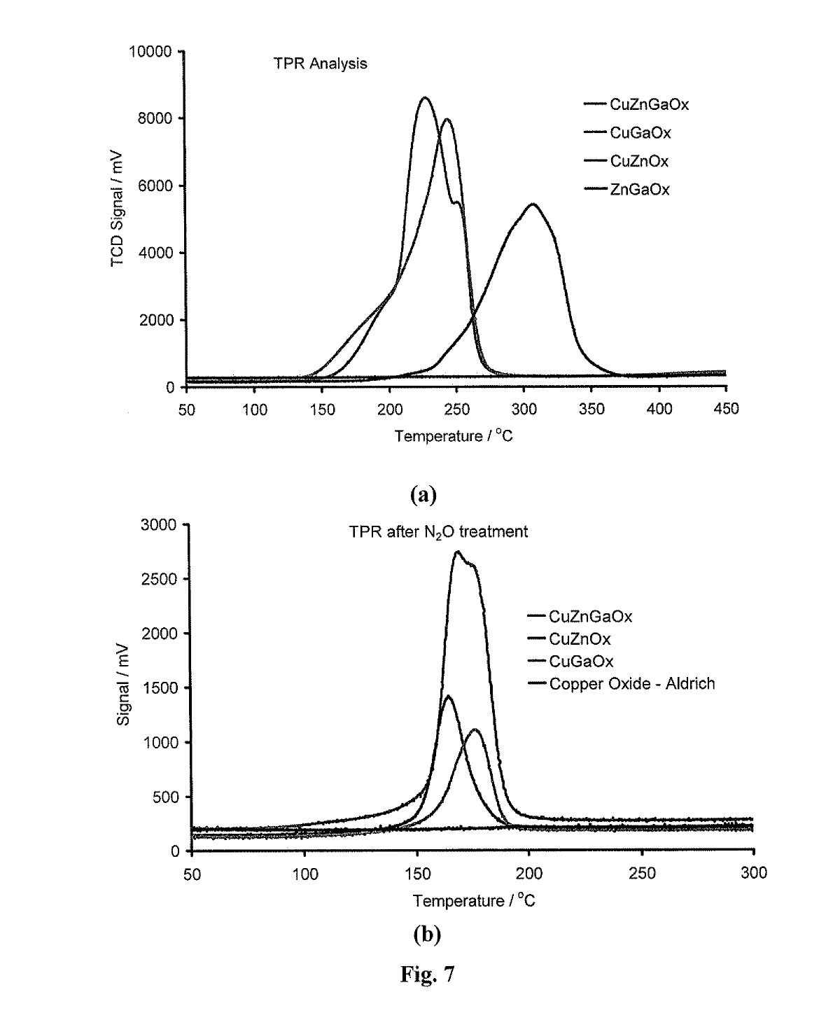

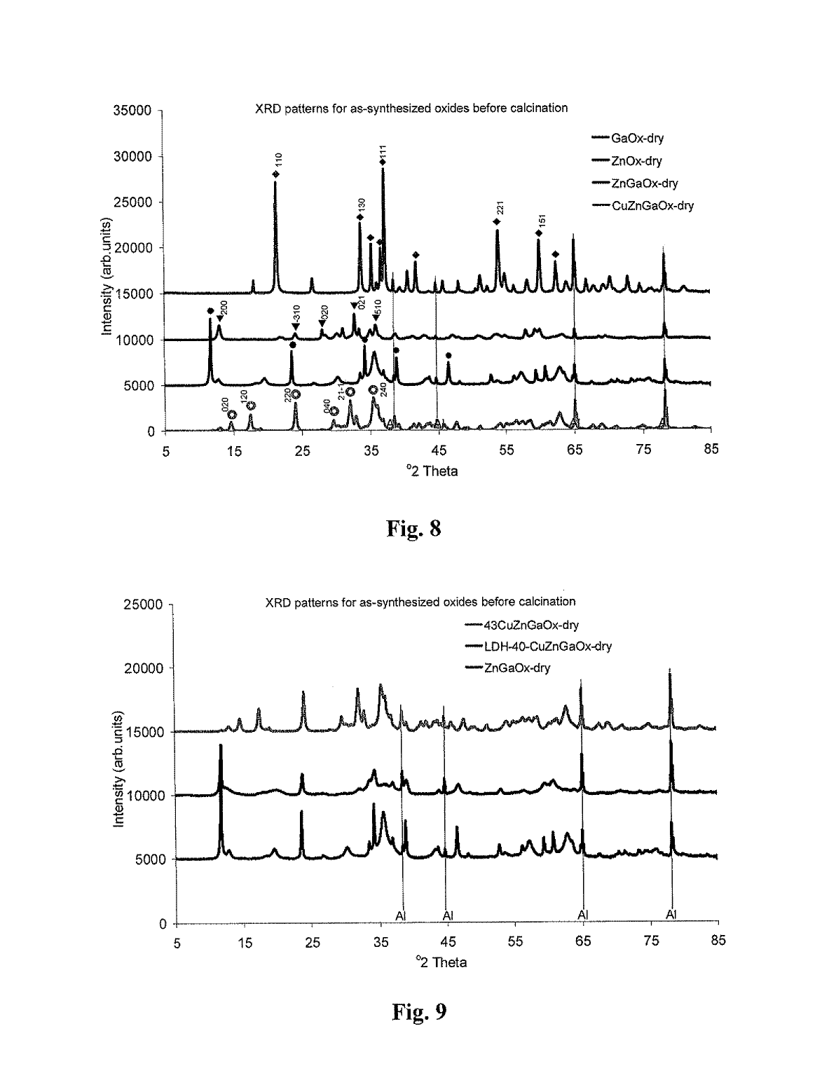

It is interesting to reveal from the catalyst testing that the Ga³⁺ incorporation to the CuZnOx can dramatically increase activity and selectivity for the NSGDSR reaction so that lower temperature for the reaction can be exploited. Thus, TPR analyses of the calcined samples of CuGaOx, CuZnGaOx and CuZnOx were carried out for comparison (FIG. 7a). It is noted that a significant hydrogen consumption was noticed for all the three samples and the areas measured indicate that an equivalent amount of CuO to Cu was taken place in the three samples during the TPR (ZnO, Ga₂O₃ and mixed ZnGa₂O₄ containing no Cu²⁺ did not give significant reduction). But the hydrogen consumption peaks were at different reduction temperatures. This indicates that Cu²⁺ in CuGaOx and CuZnGaOx was clearly in different chemical environment from CuZnOx. Detailed XRD analysis in FIG. 8 suggests that the addition of Ga³⁺ to Zn²⁺ (or Cu²⁺) would facilitate hydrotalcite phase formation at mild conditions, which appears to be an essential ingredient for this solid structure. Notice that the CuZnGaOx containing the same hydrotalcite structure but in a large quantity of Cu carbonate hydrate did not allow its easy phase identification. The hydrotalcite phase became more significant when the Cu content was reduced. The solid structure can be described as layered double hydroxides (LDHs) comprising layered materials with positively charged and charge balancing anions located in the interlayer region. The LDHs are commonly represented by the formula [M²⁺₁₋ₓM³⁺ₓ(OH)₂]^{q+}(Xⁿ⁻)_{q/n}.yH₂O. Hence, the Ga³⁺ can form homogeneous solid LDHs phase with either Cu²⁺ or Zn²⁺ in the catalyst precursors. We thus show that the homogeneous dispersion of Cu²⁺ with Ga³⁺ and Zn²⁺ in LDHs is essential to generate small copper particles upon reduction. As seen from FIG. 7b, after TPR and N₂O reoxidation the CuZnGaOx sample gave the highest second TPR peak. This clearly suggests that the Ga³⁺ addition facilitate the formation of the smallest copper particles during the catalysis (Table 2), which give the highest activity for the methanol decomposition. We do not yet know the precise reason for the exciting inhibition of CO formation in the presence of Ga³⁺ under the reaction conditions. This could be attributed to the alteration of Cu⁺/Cu⁰ distribution on the working catalyst or electronic structure of the underlying oxide.⁷⁻⁹ An investigation of the desirable metal-support interaction(s) is currently underway.

TABLE 2
Physico-chemical properties of Cu catalyst acquired
using N₂O passivation method

| | | Specific Cu metal | Specific Cu metal | |
|---|---|---|---|---|
| | | surface area of the | surface area of the Cu | Cu particle |
| | | catalyst (m²/g | in the catalyst (m²/g | size, diameter |
| Sample | Dispersion (%) | cat.) | Cu metal) | (nm) |
| 43CuZnGaOx | 30.2 | 78.4 | 221.6 | 3.0 |
| 43CuGaOx | 9.3 | 25.2 | 74.6 | 9.0 |
| 43CuZnOx | 12.6 | 31.7 | 85.4 | 7.8 |
| CuO- | 0.2 | 1.6 | 1.6 | 432.3 |
| Aldrich | | | | |

### CONCLUSION

In situ catalytic production of hydrogen by steam reforming of methanol is an attractive option for use in decentralised production of clean electrical energy from PEM fuel cells. Present technology for methanol reformations including steam reforming, partial oxidation and their combination via syn-gas route suffer from problems that would require cumbersome CO cleanups otherwise leading to severe degradation in fuel cells performance. On the other hand, there is no sufficient room for such operation for portal fuel cell consumer products. Here we report a new non-syngas direct steam reforming (NSGDSR) route at <200° C. that can integrate the endothermic methanol steam reforming with the exothermic heat generated from the PEM fuel cells which may offer good heat transfer characteristics.

In addition, blending oxygen/air to NSGDSR to encourage combustion may also balance the total thermodynamic requirements of this system. The important finding from this work is that under this reaction condition, no CO formation (<10 ppm) is observed while a high conversion of methanol to CO₂/H₂ is evident. As a result, this new route can give high quality of hydrogen for the mobile fuel cells. In addition, we have identified active type of catalysts basing on CuGaZnOx which can deliver high activity and selectivity for hydrogen production from the NSGDSR route. Evidence suggests the Ga³⁺ incorporation in Cu/ZnO system renders the formation of a homogeneous LDHs solid structure where ultrafine copper particles can be generated from this solid precursor. It appears that it is an essential step to produce high active and selective catalyst for this reaction due to desirable metal-support interactions.

### Example 2

### Rationalising the Behaviour of Cu/Zn/Ga Oxide Catalysts in Low Temperature Steam Reforming of Methanol

Having identified 43% Cu—ZnGaOₓ, as a new high performance catalyst, the focus of this project was to investigate differences between the catalysts containing differing levels of Cu, Zn, Ga and combinations thereof. It was decided that the comparisons should be made between the activities observed at 195° C. rather than 150° C., because the differences in MeOH conversion and CO production were more pronounced. To this end, a variety of characterisation techniques were employed to elucidate the structural & mechanistic properties of the catalyst, and comparisons were made by varying the support composition and changing the Cu-loading. Table 3 shows the difference in activity for a range of Cu-based catalysts:

TABLE 3
Activity data for Cu-based catalysts on Zn/Ga supports

| Catalyst | MeOH Conversion (%) | CO Concentration (ppm) |
|---|---|---|
| Johnson Matthey | 29.3 | 251 |
| HiFUELT R120 catalyst | | |
| 15% Cu—ZnGaOₓ | 8.3 | 117 |
| 43% Cu—ZnGaOₓ | 33.4 | 108 |
| 60% Cu—ZnGaOₓ | 29.1 | 176 |
| 43% Cu—ZnO | 3.7 | 423 |
| 43% Cu—Ga₂O₃ | 15.4 | 95 |

### Experimental

### Synthesis

The catalysts were prepared via a co-precipitation method. The precursor metal nitrate salts were dissolved and combined in 100 ml de-ionised water, with aqueous Na₂CO₃ (3.50 g in 100 ml DI water) used to produce the precipitate. Both solutions were added at 0.05 ml s−1 to a round-bottomed flask containing 300 ml DI water and a magnetic stirring bar maintained at 1,500 rpm, and heated to 80-90° C. with pH controlled between 6-7. The resulting precipitate was aged in solution at this temperature for 24 hrs. After ageing, the precipitate was centrifuged 6-12 times at 6,000 rpm for 5 min and washed with 50 ml DI water between each period to remove Na+ ions. The resulting solid was powdered, then dried in air at 80-100° C. overnight and subsequently calcined in static air at 3° C. min−1 up to 380° C. for 3 hrs to produce the catalyst.

TABLE 4
Quantity of precursor salts required to produce catalysts
with the desired molar ratios

| | Cu(NO₃)₂• | Zn(NO₃)₂• | Ga(NO₃)₂• | Cu:Zn:Ga |
|---|---|---|---|---|
| Catalyst | 3H₂O/g | 6H₂O/g | xH₂O/g | Molar Ratio |
| 15% | 0.54 | 2.40 | 2.15 | 3:8.5:8.5 |
| Cu—ZnGaOₓ | | | | (=15% mol. |
| | | | | Cu) |
| 43% | 3.03 | 2.40 | 2.15 | 3:2:2 |
| Cu—ZnGaOₓ | | | | (=43% mol. |
| | | | | Cu) |
| 60% | 3.03 | 1.20 | 1.08 | 3:1:1 |
| Cu—ZnGaOₓ | | | | (=60% mol. |
| | | | | Cu) |
| 43% | 3.03 | 4.80 | 0 | 3:4:0 |
| Cu—ZnO | | | | |
| 43% | 3.03 | 0 | 4.30 | 3:0:4 |
| Cu—Ga₂O₃ | | | | |
| ZnO | 0 | 2.40 | 0 | 0:1:0 |
| ZnGaOₓ | 0 | 4.80 | 4.30 | 0:1:1 |
| Ga₂O₃ | 0 | 0 | 2.15 | 0:0:1 |

Table 4 summarises the quantities of precursor salt required to synthesise catalysts with the desired molar ratios. The realatomic content of Cu, Zn and Ga in the catalysts was calculated according to the actual amount of precursor salts added, which sometimes deviated slightly from the desired quantity. Table 5 summarises the catalysts prepared using the co-precipitation method that underwent further characterisation:

TABLE 5
Summary of catalysts that underwent further characterisation

| | Precursor Salt/atomic % | | |
|---|---|---|---|
| Catalyst | Cu | Zn | Ga |
| 15% Cu—ZnGaOₓ | 14.5 | 52.2 | 33.3 |
| 43% Cu—ZnGaOₓ | 48.7 | 31.3 | 20.0 |
| 60% Cu—ZnGaOₓ | 65.5 | 21.0 | 13.5 |
| 43% Cu—ZnO | 43.7 | 56.3 | 0 |
| 43% Cu—ZnGaOₓ(Zn:Ga = 3:2) | 47.6 | 36.8 | 15.6 |
| 43% Cu—ZnGaOₓ(Zn:Ga = 1:3) | 51.7 | 16.6 | 31.7 |
| 43% Cu—Ga₂O₃ | 54.9 | 0 | 45.1 |

(N.B. In some cases the resulting atomic content deviated significantly from the originally desired quantity. For example, in the case of 43% Cu—ZnGaOₓ (Zn:Ga=1:3), the actual atomic content means that the catalyst should be renamed 52% Cu—ZnGaOₓ (Zn:Ga=1:2). However, for the purposes of this project, and to avoid confusion, the names shall remain as originally assigned.)

### Catalyst Testing

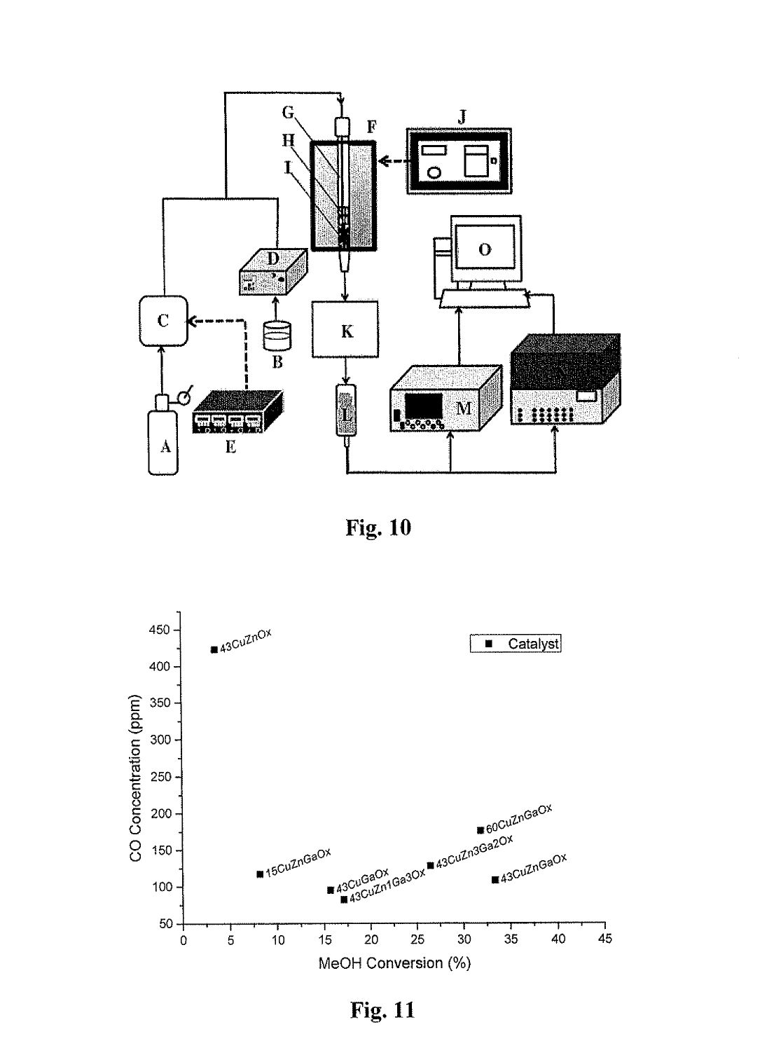

Determination of catalytic activity was carried out in a home-built plug-flow reactor system (FIG. 10).

Prior to testing, the calcined catalyst was pre-reduced using TPR from room temperature to 195° C. under 10% H₂/N₂ gas stream at 20 ml min⁻¹ for 2 hrs. Afterwards, a powder mixture of 0.40 g catalyst and 0.40 g silicon carbide (I), sandwiched between silica wool plugs, was placed in a 4 mm diameter silica reactor (G) containing a thermocouple (G), and housed in a temperature-programmable furnace (J, F). A liquid feed of CH₃OH:H₂O=1:2 (B) at a flow rate of 0.1 ml min⁻¹ generated from an HPLC pump (D) was mixed with N₂ flow (A) set at 10 ml min⁻¹ by a mass-flow controller (C, E). The mixture was allowed to pass through pre-heated tubing maintained at 195° C. where all liquids were converted to gas before reaching the reactor. After passing through a dry-ice cold trap to remove water and methanol (K, L), the product gas stream was analysed on a connected PC (O) using pre-calibrated gas chromatography with a thermal conductivity detector (M). Thus, the H₂, CO, CO₂, N₂ and CH₄ gas levels could be quantified directly. A separate FID (N) with a detection limit of CO≦10 ppm was also used.

### Characterisation Techniques

### TPR

Temperature-programmed reduction is a useful technique for studying the reducibility of solid materials. In heterogeneous catalysis, the solid catalyst powder usually exists as a precursor metal oxide under ambient conditions, which is inactive to the desired catalytic process, and so must first be ‘pre-reduced’ to produce the active material. For the low temperature SRM reaction, it is desirable to achieve the lowest possible temperature for pre-reduction of the catalyst, so that as much of the inactive metal oxide as possible is converted into the active phase, and then maintained during catalysis without deactivation due to re-oxidation or thermal sintering. Therefore by studying the reducing properties of the different Cu-based catalysts, we may begin to understand the differences between them in terms of activity.

TPR measurements were carried out on a ThermoQuest TPDRO 1100 instrument. 0.026 g of the solid powder sample was sandwiched between two tufts of glass wool inside the TPR tube, accompanied by a thermocouple, and inserted into the instrument. Helium pre-treatment (10° C. min⁻¹ at 10 ml min⁻¹ from 20-150° C., then held for 5 min before allowing to cool) was carried out first to remove any ambient gas molecules adsorbed on the catalyst surface. Then reduction of the copper oxide within the material (Eq. A) was achieved by running 5% H₂ in Argon through the TPR tube at 5 ml min⁻¹ at a temperature ramp of 2° C. min⁻¹ from 40-800° C., then held at 800° C. for 30 min before allowing to cool to room temperature.

Cu^{(II)}O+H₂→Cu⁰+H₂O (Eq. A)

The change in conductivity of the gas stream due to the consumption of hydrogen was measured as a function of both time and temperature, and the results plotted in the form of a TPR profile.

### N₂O Chemisorption

Chemisorption techniques are used in heterogeneous catalysis to investigate the properties of the active metal. Important properties such as Cu dispersion, surface area and particle size may be revealed via chemisorption methods. To determine these properties, a chemisorption technique using N₂O was carried out in a manner similar to literature methods.¹¹ 0.026 g of sample was firstly pre-treated with He, as before, then pre-reduced under hydrogen using the normal TPR method, up to 330° C. Once the sample had cooled down to room temperature, 5% N₂O/Ar at 20 ml min⁻¹ was allowed to flow through the TPR tube for 40 min in order to re-oxidise the exposed Cu only, via dissociative chemisorption (Eq. B):

N₂O_{(g)}+2Cu₍ₛ₎→Cu₂O₍ₛ₎+N_{2(g)} (Eq. B)

After N₂O treatment, He pre-treatment (10 ml min⁻¹ for 10 min at room temp.) was again carried out to remove any adsorbed N₂O, followed by 2^{nd} TPR treatment up to 330° C., with the gas stream conductivity measured as a function of time and temperature. In order to determine the Cu surface area, it was necessary to calibrate a Cu^{(II)}O standard, with known Cu content, against which the samples could be compared. TPR was performed on 0.005 g, 0.010 g and 0.015 g of Cu^{(II)}O from Aldrich, and the number of moles of hydrogen consumed was calculated via knowledge of the Cu content. From this, a simple 1^{st} order calibration was found by plotting the number of moles H₂ consumed versus the TPR integrated peak area. By comparing the TPR integrated peak area of the catalyst sample with the calibration file, it was possible to determine the Cu dispersion, specific surface area and particle size.

### Powder XRD

Powder X-ray diffraction data was obtained for catalyst materials before and after calcination in order to investigate the crystal structure of the bulk material. In general, the precursor material will be a quasi-amorphous structure composed of various carbonates and hydroxycarbonates, which release CO₂ and H₂O upon calcination to produce the more crystalline active catalyst. It is likely that the final catalyst will be composed of more than one crystal phase, and it is possible either that one of these phases will be the most active, or that a combination of different phases is in fact required for the most effective catalysis.

To obtain the diffraction peaks, a small quantity of sample was placed onto an aluminium plate, and the X-ray diffraction data was collected on a Philips PW-1729 diffractometer using a monochromated Cu Kα beam. The aluminium plate gives characteristic diffraction peaks which can be used as a reference marker against which the peaks of the material under investigation can be compared.

### AC Impedance

The impedance of a material describes its resistance to alternating current. It is represented by the complex quantity, Z, and by plotting the real component (resistance, Z₁) versus the imaginary component (reactance, Z₂) of a material's impedance across a range of frequencies we obtain a semi-circular spectrum known as a Nyquist plot, By obtaining such spectra over a range of temperatures, it is possible to gain information about the charge carrying properties of the material. In the case of the present study, the predominant mechanisms for electrical conductivity within the calcined catalyst materials are either via mobile Cu ions, in which Cu is able to move between either substitution-able lattice sites or interstices, or via oxygen mobility, where O²⁻ anions are able to ‘hop’ into adjacent vacant lattice sites. These processes have associated thermal activation energies, which describe the ease with which a mobile ion can move between sites. If we model the impedance of the material by a simple Arrhenius equation, then an Arrhenius plot of ln|Z₂∥ vs. 1/T at fixed frequency should yield a straight line of gradient Eₐ/R, from which the activation energy associated with the ionic mobility of the material may be extracted.

$${Z}_{2} = {Ae}^{- {E}_{a} / RT} \qquad ( Eq . C )$$

To obtain the impedance measurements over a range of temperatures, the calcined catalyst was pressed at 5 tonne pressure into a pellet of approx. 1 mm thickness and 30 mm diameter, then held between two platinum electrodes inside a quartz tube containing a thermocouple, and wrapped in an electrical thermal jacket attached to a programmable heating furnace. The electrodes were connected to an Ivium CompactStat electrochemical interface, which in turn was connected to a PC with the corresponding IviumSoft software. For each material studied, the sample was heated up to 220° C. and maintained at that temperature for 1 hr, then allowed to cool by 20° increments and held at each temperature for 20 min before taking a measurement. The frequency range used was from 100,000 Hz to 2 Hz, the current range was 100 μA and the frequency scan amplitude was 0.5V.

### EPR Spectroscopy

In the presence of an external magnetic field (B₀), the electrons within a material will align their magnetic moments either parallel (mₛ=−½) or anti-parallel (mₛ=+½) to the applied field. These alignments have different energies, and the phenomenon is known as the Zeeman effect.

The separation between the energy states can be written in terms of the g-factor (gₑ) and the Bohr magneton (μ_{B}):

$$\begin{matrix}Δ E = {E}_{+ \frac{1}{2}} - {E}_{- \frac{1}{2}} \\ = {g}_{e} {μ}_{B} {B}_{0}\end{matrix} \qquad ( Eq . D )$$

Unpaired electrons within the material can move between the energy levels by absorbing a photon that satisfies the resonance condition (Eq. E):

hν=gₑμ_{B}B₀ (Eq. E)

The statistical distribution of unpaired electrons within a paramagnetic sample is described by the Boltzmann distribution (Eq. F):

$$\frac{{n}_{upper}}{{n}_{lower}} = {e}^{- \frac{hv}{kT}} \qquad ( Eq . F )$$

In practice there will be a slightly larger population in the lower energy state than the upper one. This means that in an external magnetic field there will be a net absorption of energy as transitions from the lower to upper state are more probable. This net absorption is measurable and forms the basis of EPR spectroscopy. The g-factor depends not only on the external magnetic field, but also local fields within the material, therefore by measuring the g-factor it is possible to investigate species in different electronic environments. The electronic environment of paramagnetic transition metal ions such as Cu²⁺ in the Cu/Zn/Ga oxide system may be investigated using EPR. By looking at the electronic environments of the Cu ions, we may be able to distinguish between different lattice sites, and possibly identify the most active site for the steam reforming reaction. Samples were analysed in a Bruker EM/Y X-band CW spectrometer using 100 mg solid sample in a quartz tube.

### Results

### Catalytic Activity

Table 6 summarises the activity and conversion properties of the synthesised catalysts, tested at 195° C., 0.1 ml min⁻¹ feed rate with a 1:2 methanol/water molar ratio. The accompanying activity scatter plot is shown in FIG. 11:

TABLE 6
Summary of SRM activity of synthesised catalysts
(the catalyst names are abbreviated)

| Catalyst | MeOH Conversion (%) | CO Concentration (ppm) |
|---|---|---|
| 15CuZnGaOₓ | 8.2 | 117 |
| 43CuZnGaOₓ | 33.4 | 108 |
| 60CuZnGaOₓ | 31.9 | 176 |
| 43CuZnOₓ | 3.6 | 423 |
| 43CuZn3Ga2Oₓ | 26.5 | 128 |
| 43CuZn1Ga3Oₓ | 17.1 | 82 |
| 43CuGaOₓ | 15.7 | 95 |

The CO concentration was measured directly via calibrated GC and FID. Methanol conversion was calculated according to the following equation (Eq. G):

$$Methanol conversion ( \% ) = \frac{\{ {[ MeOH ]}_{in} - {[ MeOH ]}_{out} \}}{{[ MeOH ]}_{in}} \times 100 \qquad ( Eq . G )$$

The results show that, once again, we observe a wide range of catalytic behaviour. As before, 43CuZnGaOₓ was the most active catalyst, achieving 33.4% MeOH conversion with only 108 ppm CO concentration. 60CuZnGaOₓ achieved similar conversion (31.9%), but CO production was significantly higher at 176 ppm. 43CuZn1Ga3Oₓ and 43CuGaOₓ achieved lower CO production, but MeOH conversion was significantly reduced. 43CuZnOₓ performed poorly, achieving only 3.6% conversion with 423 ppm CO production.

### TPR

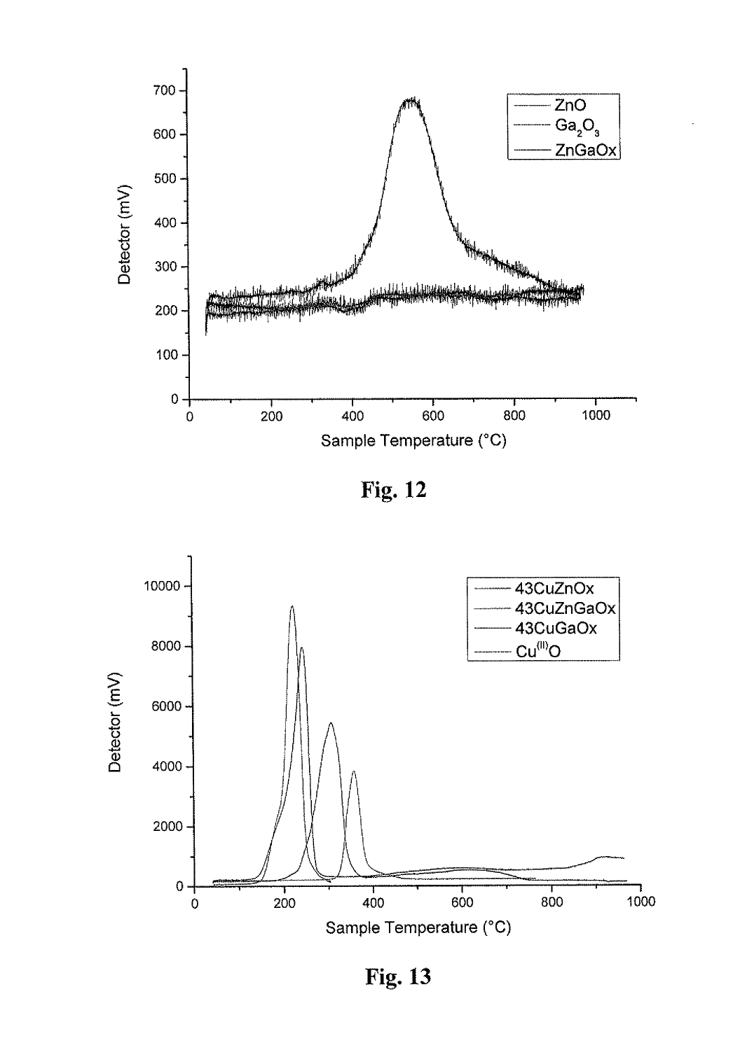

The TPR profiles of a range of Zn/Ga oxides with and without Cu were obtained over the temperature range 40-800° C. Both the Zn/Ga ratio and the Cu-loading were varied in order to investigate the change in reducibility. The following profiles show the change in gas stream conductivity as a function of temperature. FIG. 12 displays the TPR profiles of ZnO, Ga₂O₃ and ZnGaOₓ. Neither ZnO nor Ga₂O₃ are reduced in the temperature range, but ZnGaOₓ shows a small but significant reduction peak at ˜550° C., which indicates the more facile reduction of Zn²⁺ species within the ZnGaOₓ structure, which will be accompanied by the formation of oxygen vacancies. The profiles for the corresponding Cu-containing oxides are shown in FIG. 13.

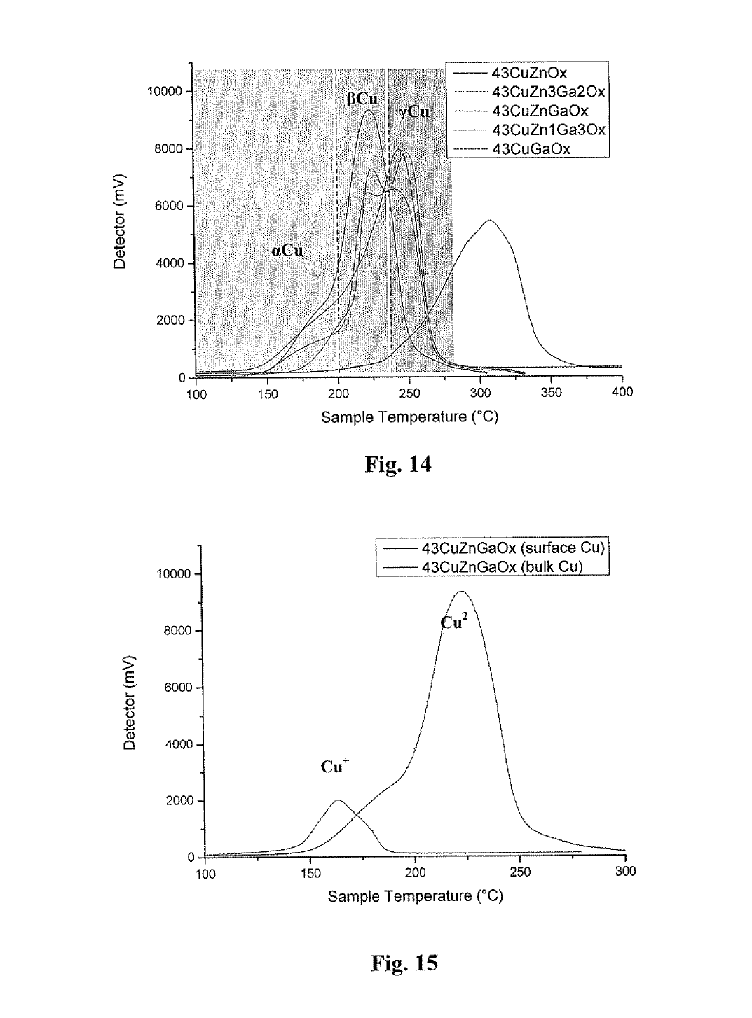

Here we observe much more significant reduction peaks, appearing at lower temperature, corresponding to the reduction of copper species within the materials. For 43CuZnO, the reduction begins at −200° C. and reaches its maximum at 300° C. For both 43CuGaOₓ and 43CuZnGaOₓ, reduction begins earlier at ˜150° C., reaching a single maximum at 220° C. in the case of 43CuZnGaOₓ, and 250° C. for 43CuGaOₓ. For both 43CuGaOₓ and 43CuZnGaOₓ we observe an initial gradual reduction, followed by a ‘kink’ at ˜200° C. where the reduction peak suddenly becomes steeper. In all cases the total peak area was the same after extensive reduction, indicating that all the Cu in the materials had been reduced. The TPR profile for 0.005 g Cu^{(II)}O is shown for reference; it can be seen that Cu reduction in the Zn/Ga systems takes place at significantly lower temperature than for CuO. FIG. 14 shows in more detail the effect of varying the Zn/Ga ratio.

From FIG. 14 it is clear that there are three distinct Cu environments present in the Cu/Zn/Ga systems (labelled αCu, βCu and γCu). αCu corresponds with the initial shallow reduction slope, which begins at ˜150° C. βCu corresponds with the ‘kink’ in the slope at ˜200° C., rising to a maximum at 220° C. γCu corresponds with the second maximum at 250° C. We can see that the onset of reduction shifts to lower temperature as Ga content is increased until 43CuZnGaOₓ, which has the largest αCu peak amplitude at the reaction temperature (195° C.), which may explain its superior activity. 43CuGaOₓ is in fact the most easily reducible, with the onset of reduction occurring earlier than any other catalyst. We might therefore have expected 43CuGaOₓ to display higher activity than the other catalysts. However, at the reaction temperature, there may be significant Cu sintering occurring which reduces the specific surface area of the copper. The results from the N₂O chemisorption will show that this is indeed the case. All of the Ga-containing systems display an αCu peak. 43CuZnGaOₓ displays the largest peak for βCu, with no observable γCu peak. The other Zn/Ga ratios display both βCu and γCu peaks, whilst 43CuGaOₓ contains the largest γCu peak but no βCu. 43CuZnOₓ contains no reducible Cu at the reaction temperature, which explains its poor SRM activity. Its reduction peak is at much higher temperature, indicating that Cu is in an entirely different environment from the Ga-containing systems. FIG. 15 shows the TPR profiles obtained from reducing bulk Cu compared with surface Cu⁺ in 43CuZnGaOₓ (via N₂O chemisorption method). From the N₂O chemisorption method, it was possible to obtain the TPR profile for the reduction of surface Cu⁺ on the catalyst surface. Reduction of surface Cu⁺ takes place at ˜150° C., reaching a maximum at 160° C. This seems to correspond with the αCu environment identified earlier. One can therefore conclude that the αCu environment consists of Cu⁺ ions on or near the catalyst surface. βCu and γCu correspond with Cu²⁺ ions in different environments in the bulk structure.

### N₂O Chemisorption

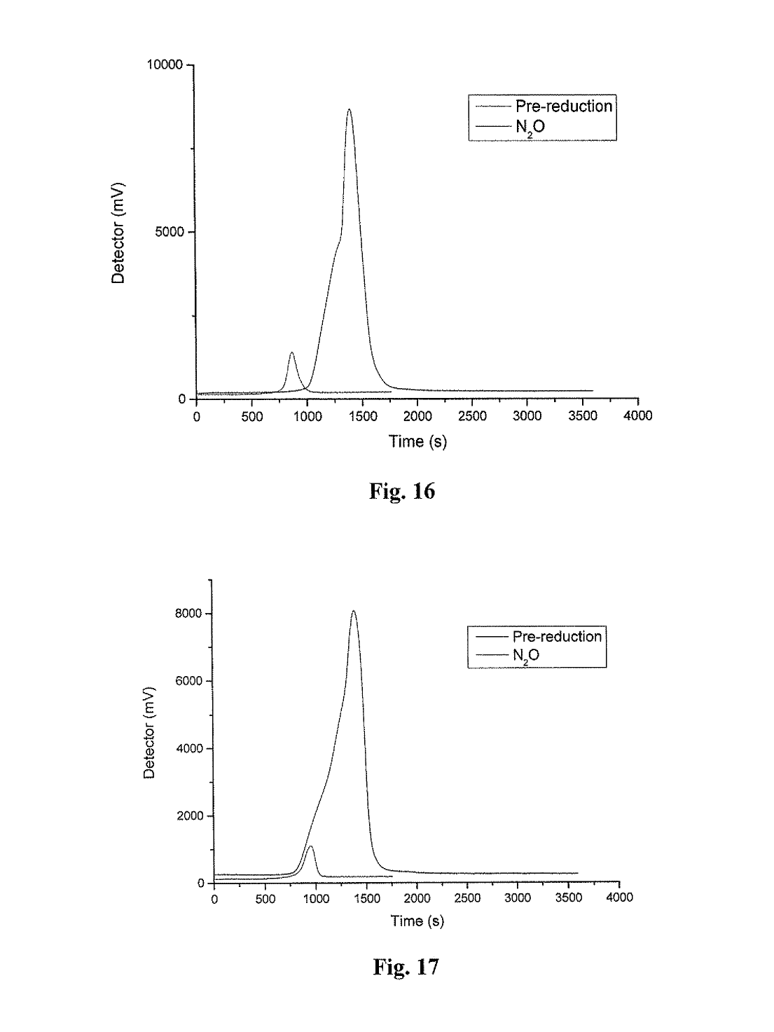

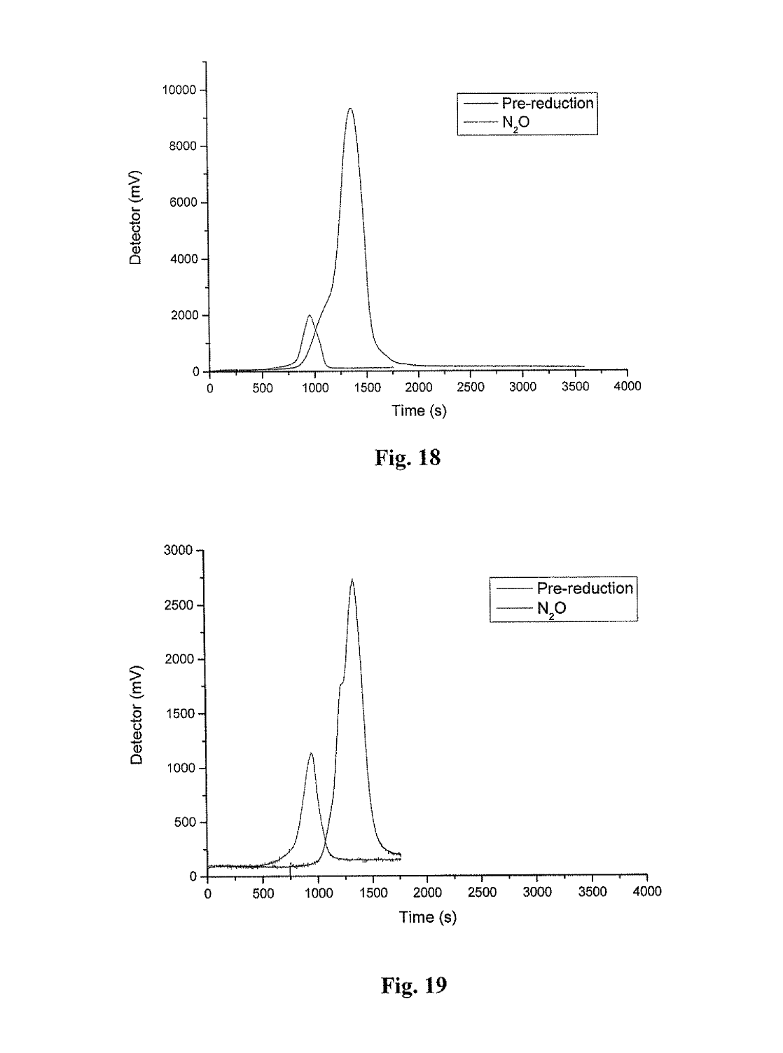

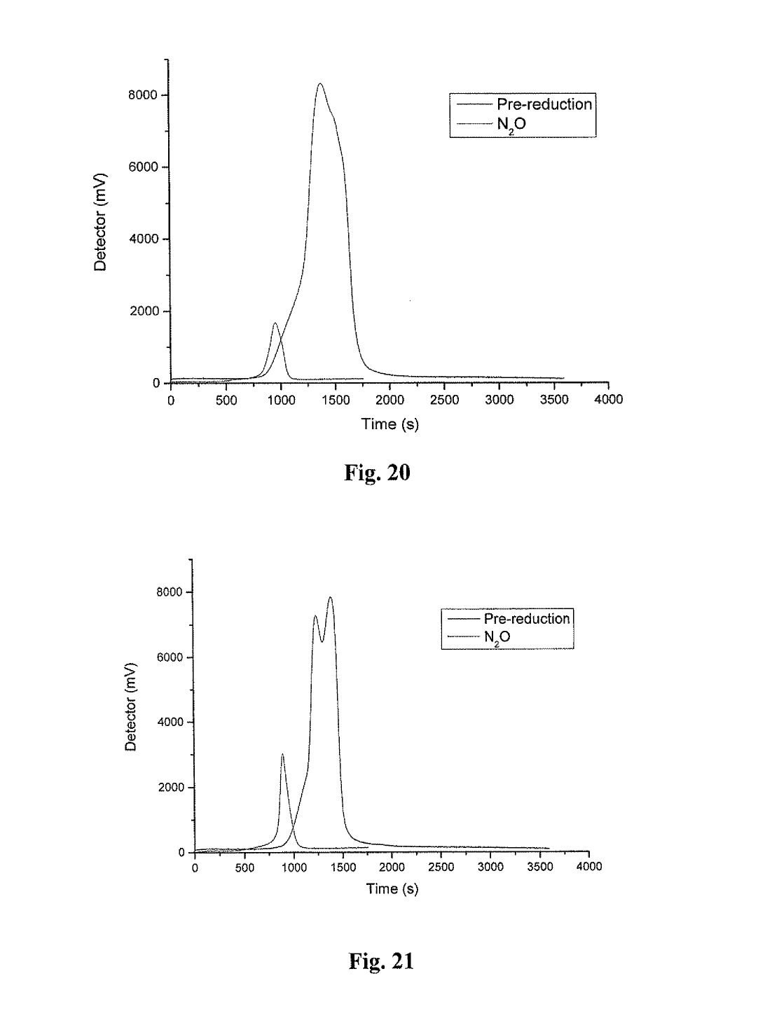

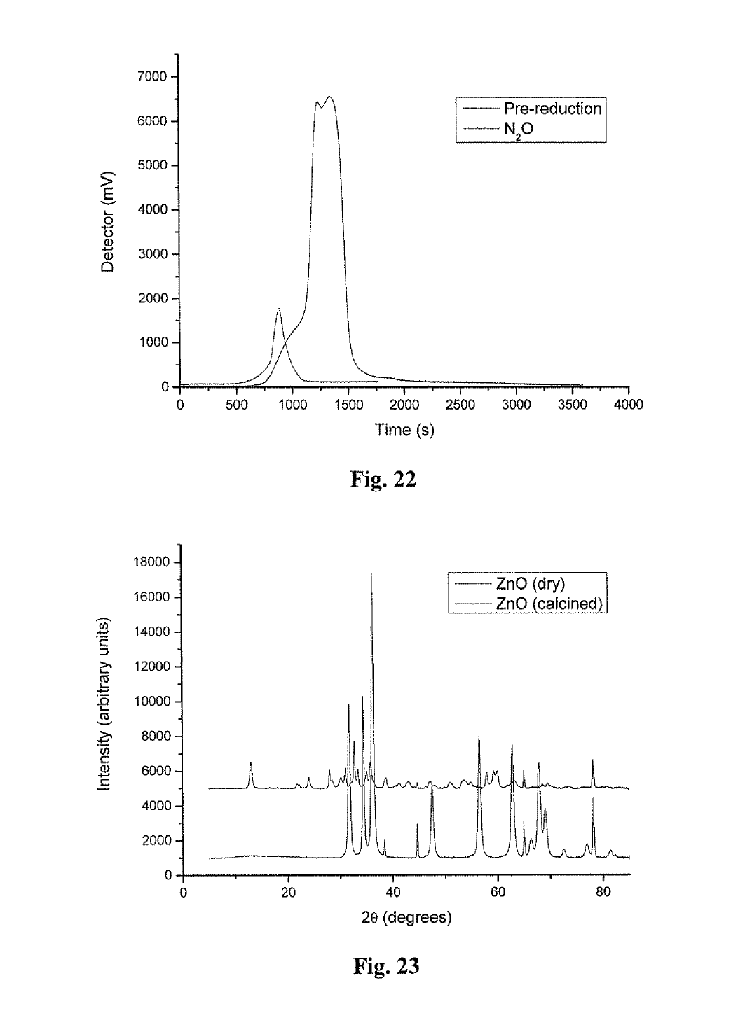

The N₂O chemisorption data was obtained for a range of Cu-containing Zn/Ga oxide materials, with the TPR profiles before and after N₂O treatment displayed as a function of time rather than temperature, since reference to the calibrated Cu^{(II)}O standard for surface area calculations was more reliable using this method. FIG. 16 shows the TPR profiles for 43CuZnOₓ, firstly during the initial reduction, then after re-oxidation at room temp. with N₂O. The pre-reduction step involves the reduction of both surface and bulk Cu in the material, whilst the second TPR after N₂O treatment only involves reduction of surface Cu₂O. The 2^{nd} TPR reductional ways took place at lower temperature, reflecting the greater reducibility of surface Cu⁺. FIGS. 17-18 show the results for 43CuGaOₓ and 43CuZnGaOₓ. FIGS. 19-20 show the results for 15CuZnGaOₓ and 60CuZnGaOₓ. FIGS. 21-22 show the results for 43CuZn3Ga2Oₓ and 43 CuZn1Ga3Oₓ.

TABLE 7
Summary of the results from N₂O chemisorption

| | | Specific Cu | Specific Cu | |
|---|---|---|---|---|
| | | metal surface | metal surface | Cu |
| | Cu | area of the | area of Cu in | particle |
| | Dispersion | catalyst | the catalyst | size |
| Sample | (%) | (m² g⁻¹ cat.) | (m² g⁻¹ Cu) | (nm) |
| 15CuZnGaOₓ | 54.8 | 33.8 | 298.1 | 2.2 |
| 43CuZnGaOₓ | 24.6 | 76.7 | 214.0 | 3.6 |
| 60CuZnGaOₓ | 14.3 | 58.4 | 110.5 | 6.7 |
| 43CuZnOₓ | 12.6 | 31.7 | 85.4 | 8.0 |
| 43CuZn3Ga2Oₓ | 29.1 | 75.9 | 210.2 | 3.58 |
| 43CuZn1Ga3Oₓ | 22.2 | 55.2 | 156.5 | 5.25 |
| 43CuGaOₓ | 9.3 | 25.9 | 74.6 | 11.7 |
| Cu^{(II)}O- | 0.2 | 1.6 | 1.6 | 432.3 |
| Aldrich | | | | |

The Cu dispersion is defined as the fraction of Cu atoms exposed to the surface. It was calculated as follows:

$$\begin{matrix}{D}_{Cu} = \frac{{N}_{Surface}}{{N}_{Total}} \\ = \frac{Surface Cu {H}_{2} Consumption ( 2 nd TPR )}{Total Cu {H}_{2} Consumption ( 1 st TPR )} \times 100 \%\end{matrix} \qquad ( Eq . H )$$

The specific Cu metal surface area of the catalyst was calculated as:

$${SA}_{cat} = \frac{Surface Cu atoms {g}^{- 1} cat .}{Surface Cu packing density} ( Units = {m}^{2} {g}^{- 1} cat . ) \qquad ( Eq . I )$$

The specific Cu metal surface area of Cu in the catalyst was calculated as:

$${SA}_{Cu} = \frac{{SA}_{cat}}{Cu loading of catalyst} ( Units = {m}^{2} {g}^{- 1} Cu ) \qquad ( Eq . J )$$

The Cu particle size was calculated as being the average diameter of the Cu particles on the surface, assuming spherical geometry:

$${x}_{Cu} ( nm ) = \frac{6 \times {10}^{9}}{{SA}_{Cu} \times Cu density} \qquad ( Eq . K )$$

(NB. The above calculations are valid only by making the assumption that N₂O is decomposed to N₂, with the simultaneous oxidation of surface Cu to Cu₂O.)

There are several trends in Table 7 that are worth highlighting. Firstly, we can see that both D_{Cu} and SA_{Cu} decrease as the Cu-loading is increased. This is reflected in the increasing Cu particle size as Cu-loading increases. However, SA_{cat} is at its maximum at 43% Cu-loading. Secondly, we observe that as the Zn/Ga ratio is varied, D_{Cu} gradually increases as Ga content is increased until a maximum for 43CuZnGaOₓ, which contains 50% Cu, 30% Zn and 20% Ga. This is accompanied by increasing SA_{Cu} and SA_{cat} and decreasing particle size. However, as Ga content is increased beyond 20% the dispersion, SA_{Cu} and SA_{cat} decrease again and the particle size increases.

### Powder XRD

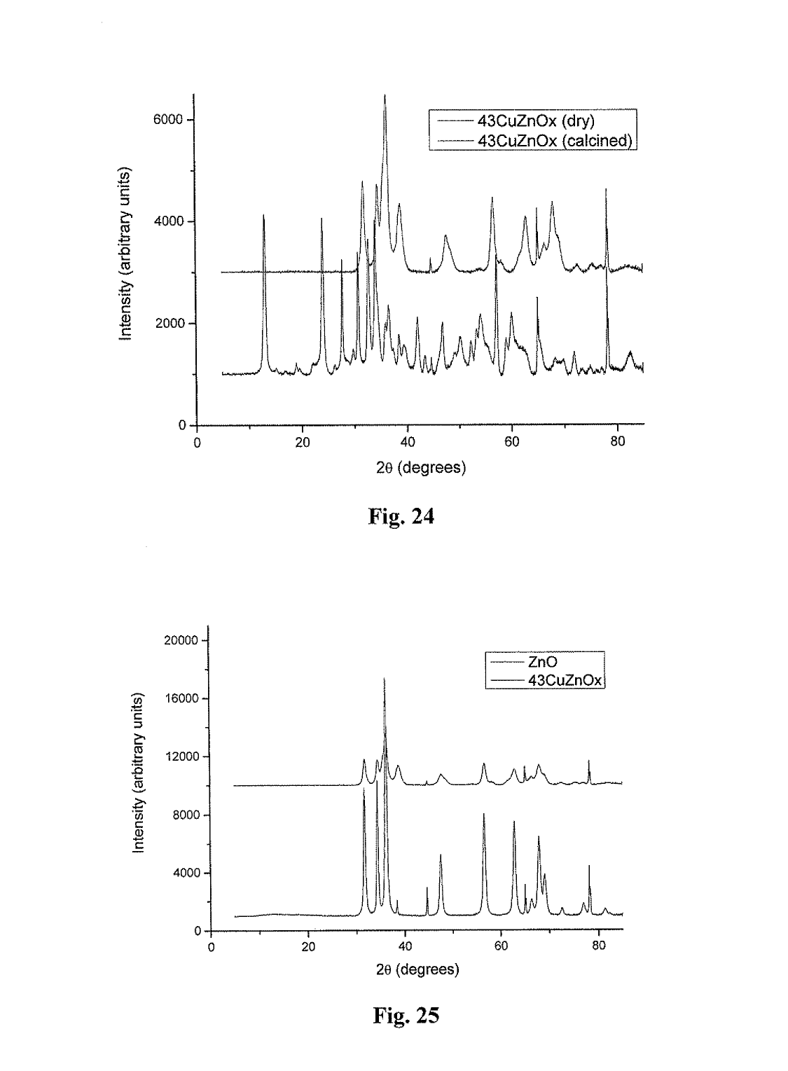

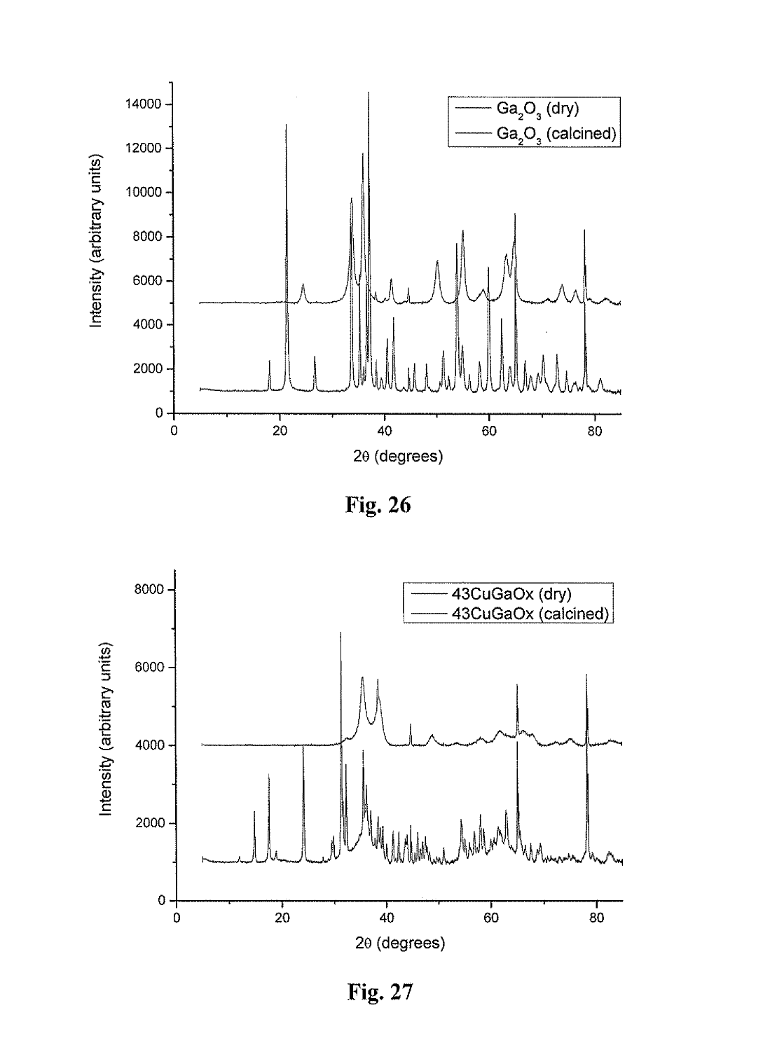

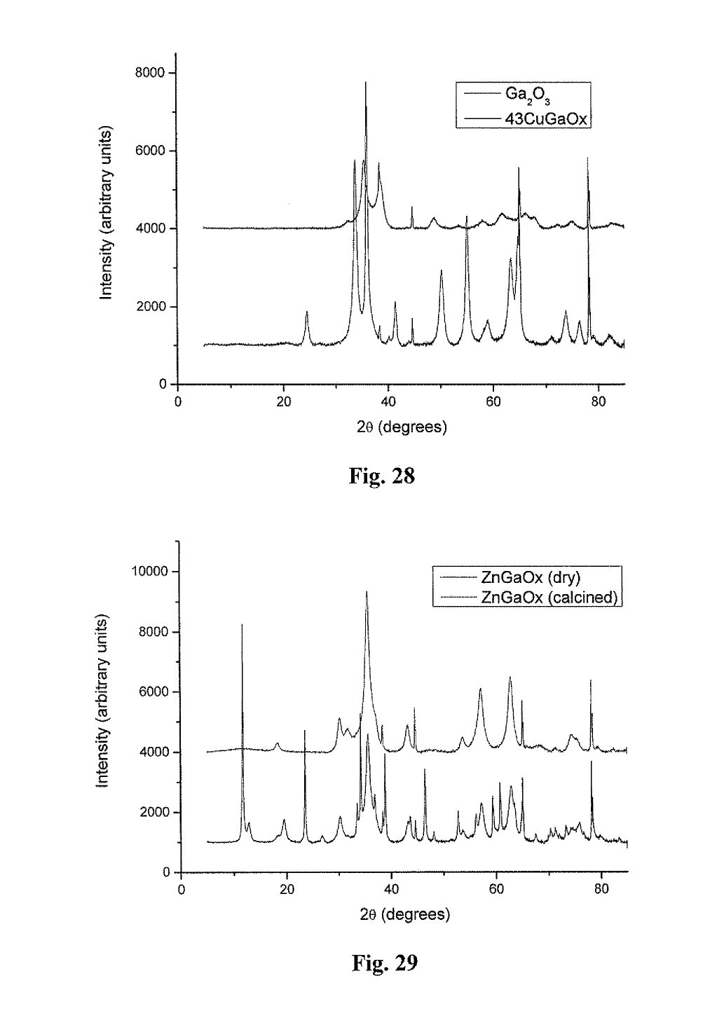

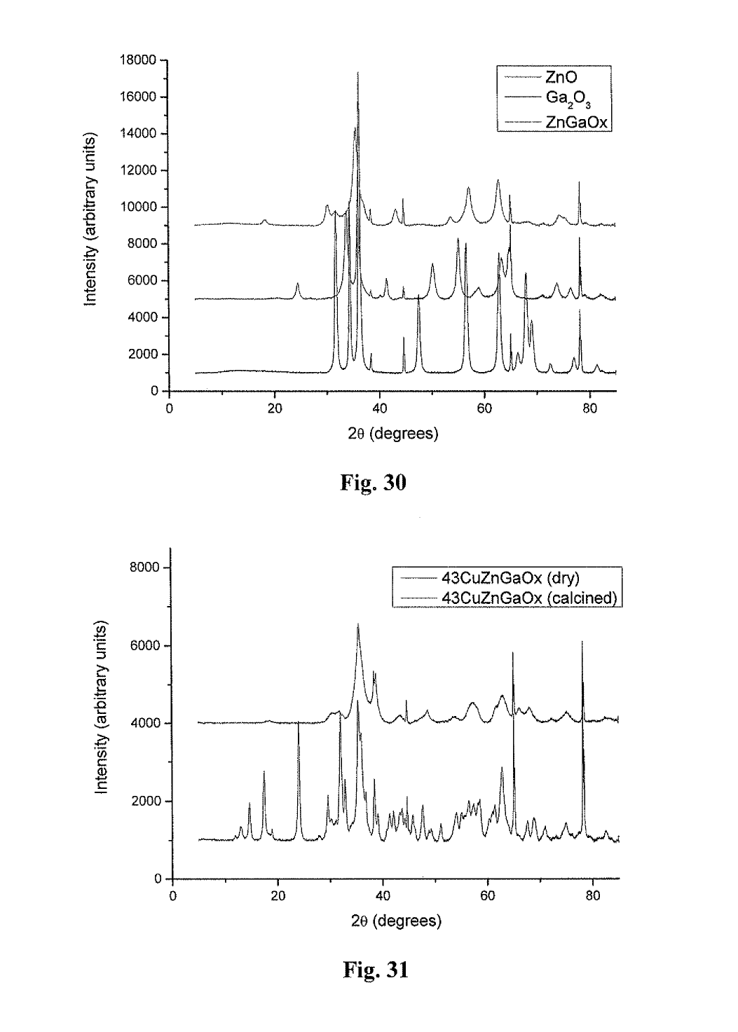

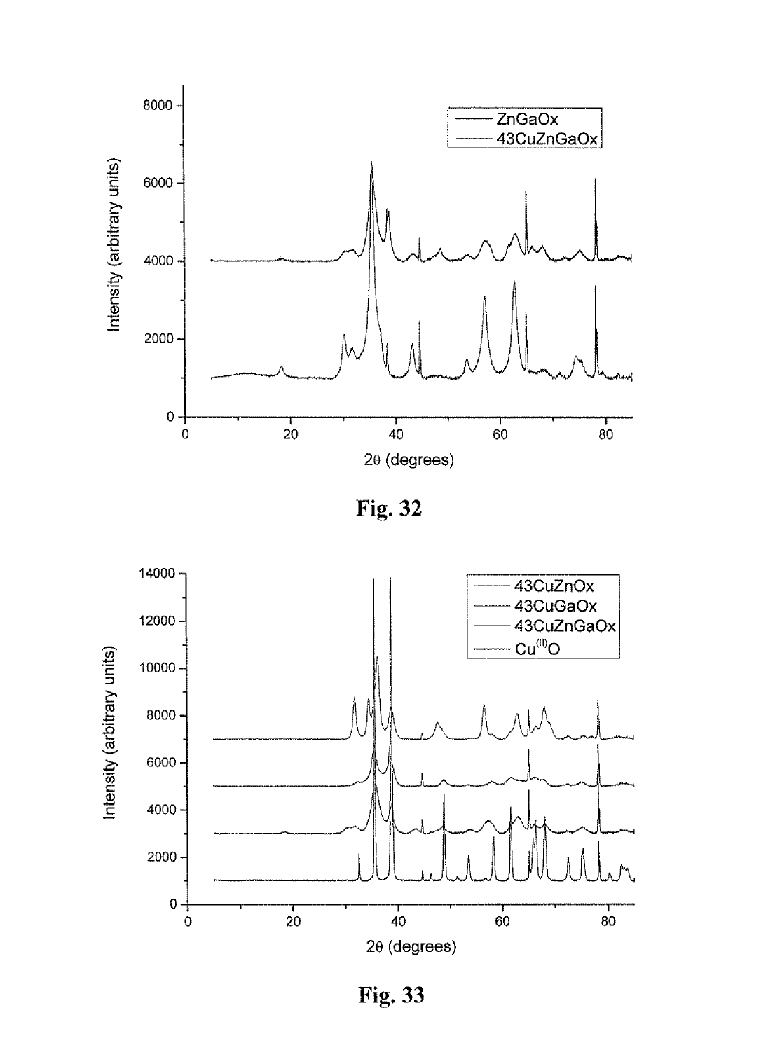

FIG. 22 shows the X-ray diffraction peaks for ZnO before & after calcination at 380° C.: The ZnO shows clearly defined peaks after calcination, indicating the more defined crystal structure of the calcined material compared with the dry precursor. The calcined ZnO diffraction peaks can be indexed to the hexagonal wurtzite structure. The Al peaks are at 38°, 45°, 65° and 78°. FIG. 24 shows the diffraction peaks for 43CuZnOₓ. FIG. 25 shows a comparison between calcined ZnO and 43CuZnOₓ. It can be seen from the comparison in FIG. 25 that the addition of Cu to the ZnO system does not alter the wurtzite structure, with no additional peaks observed, although the wurtzite peaks are reduced in intensity. This indicates that the material exists as a homogeneous solid solution of Cu/ZnO. FIG. 26 shows the X-ray diffraction peaks for Ga₂O₃. The precursor peaks are much more defined for Ga₂O₃ than for ZnO, but once again the structure resolves into a more crystalline form upon calcination. The calcined Ga₂O₃ diffraction peaks can be indexed to rhombohedral α-Ga₂O₃. FIG. 27 shows the diffraction peaks for 43CuGaOₓ. FIG. 28 shows a comparison between calcined Ga₂O₃ and 43CuGaOₓ. This time it is clear that the presence of Cu significantly alters the structure; the α-Ga₂O₃ phase is no longer present in 43CuGaOₓ. The addition of Cu triggers the formation of a poorly crystalline CuGa₂O₄ tetragonal spinel phase, as shown in FIG. 27. CuO also appears to be present. FIG. 29 shows the diffraction peaks for ZnGaOₓ. The majority of the diffraction peaks for calcined ZnGaOₓ can be indexed to a cubic spinel structure, but it can also be seen from the comparison in FIG. 30 that wurtzite ZnO and α-Ga₂O₃ phases are both present; therefore ZnGaOₓ consists of a heterogeneous mixture of various Zn/Ga oxide phases. FIG. 31 shows the XRD data for 43CuZnGaOₓ. Both the dry and calcined 43CuZnGaOₓ bear resemblance to the peaks obtained for ZnGaOₓ, indicating that the predominant cubic spinel phase is maintained upon the addition of Cu. The similarities can be seen in FIG. 32. A comparison between the calcined Cu-containing materials is displayed in FIG. 33. The diffraction pattern contains unique diffraction peaks corresponding with the cubic spinel phase. There is also evidence that the tetragonal spinel phase identified for 43CuGaOₓ is also present in 43CuZnGaOₓ. The diffraction peaks for Cu^{(II)}O are also shown, and it can be seen that many of the CuO peaks overlap with those that were previously indexed. It is likely that CuO will be present as part of a heterogeneous mixture in all of the catalysts prepared, especially if the stoichiometry does not fit with the already identified phases. Excess Cu may also be present in interstitial sites within one or more of the phases.

### AC Impedance

The AC impedance spectroscopy results were obtained, in air, for a range of catalyst and metal oxide materials pre-calcined in air at 330° C. The data obtained for each material are summarised in the table below.

TABLE 8
AC impedance results summary

| | Resistance (Z₁) at | | Temperature Range |
|---|---|---|---|
| Catalyst | 200° C. (Ω) | Eₐ (eV) | (° C.) |
| 43CuZnOₓ | 4,000 | 0.514 | 30-220 |
| 43CuZnGaOₓ | 7,000 | 0.661 | 80-220 |
| 43CuGaOₓ | 14,000 | 1.102 | 120-220 |
| ZnO | 800,000 | 0.979 | 200-220 |
| ZnGaOₓ | >10⁶ | 0.808 | 260-300 |
| β-Ga₂O₃ | >10⁶ | ~1.1 | 167-300 |

From Table 8 it can be seen that the conductivity of the Cu-containing materials is greatly enhanced by several orders of magnitude relative to their non-Cu analogues, with a concomitant lowering of the thermal activation energy. This can be attributed to the presence of mobile Cu ions, which act as the dominant charge carrier. For the non-Cu analogues the dominant charge carrying mechanism is via O-vacancies, but the activation energy for the migration of vacancy lattice defects is much higher than for mobile Cu ions. The addition of Ga to these systems causes a decrease in the conductivity, the reasons for which will be explained in the discussion section.

### EPR Spectroscopy

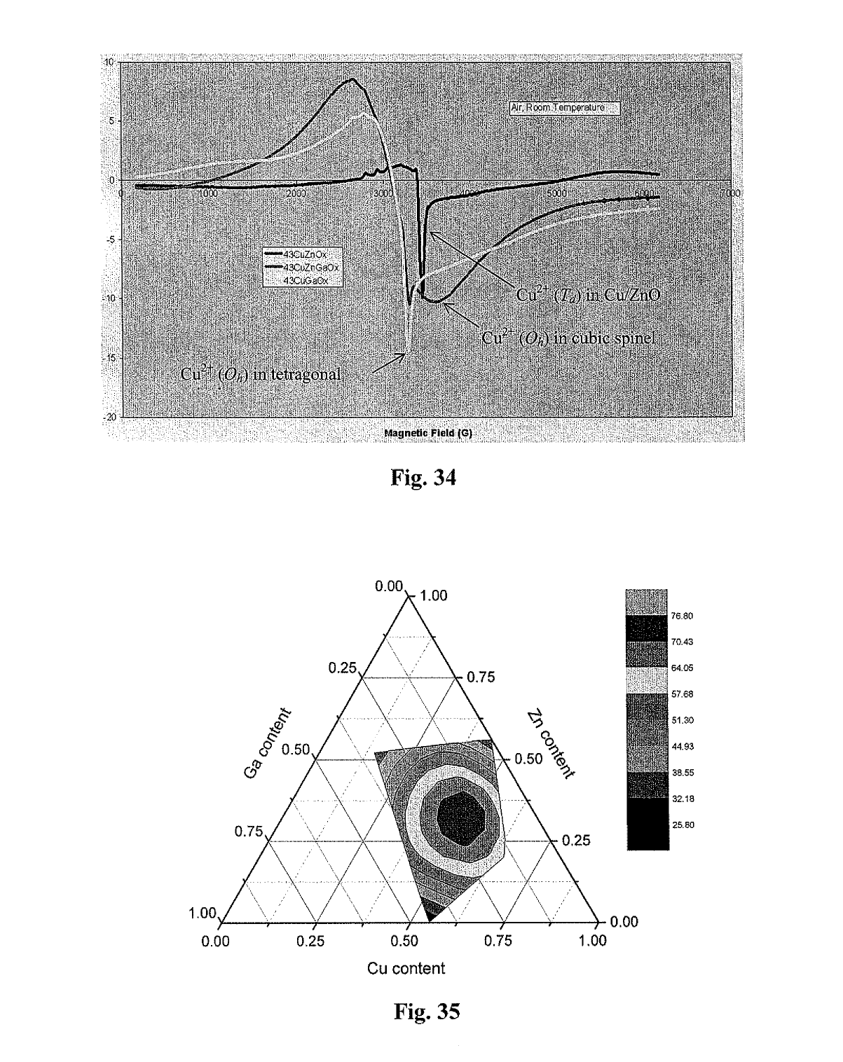

The EPR spectra obtained with 43CuZnOₓ, 43CuZnGaOₓ and 43CuGaOₓ are shown in FIG. 34. 43CuZnOₓ displays a strong, sharp signal at 3,500 G, corresponding with Cu²⁺ ions in a tetrahedral environment within the hexagonal wurtzite Cu/ZnO solid solution. There are also several small ‘bumps’ at ˜3,000 G corresponding with isolated superficial Cu ions on the surface. In 43CuGaOₓ a strong signal can be observed at 3,200 G. This peak corresponds with Cu²⁺ ions in the CuGa₂O₄ tetragonal spinel phase. In 43CuZnGaOₓ this signal is still present indicating the existence of the tetragonal spinel phase within the 43CuZnGaOₓ structure, as proposed from the XRD. There is also a broad signal centred at ˜3,600 G. This peak most likely corresponds with Cu²⁺ ions in an octahedral environment within the cubic spinet structure identified from the XRD. Cu²⁺ ions in CuO are EPR silent, and any Cu⁺ present in the catalysts will not give an EPR signal because they are not paramagnetic.

### Discussion

The catalytic properties of a material are linked to its structure, since the structure plays an important role in dictating the surface area, the nature and availability of active sites and the strength of metal-support interactions. Table 9 summarises all of the main results from Example 2.

TABLE 9
Summary of results in Example 2

| | | | | MeOH | CO | | | | | |
|---|---|---|---|---|---|---|---|---|---|---|
| | Atomic Content (%) | | | Conv. | Conc. | D_{Cu} | SA_{cat} | SA_{Cu} | x_{Cu} | Eₐ |
| Catalyst | Cu | Zn | Ga | (%) | (ppm) | (%) | m² g⁻¹ | m² g⁻¹ | (nm) | (eV) |
| 15CuZnGaOₓ | 14.5 | 52.2 | 33.3 | 8.2 | 117 | 54.8 | 33.8 | 298.1 | 2.2 | — |
| 43CuZnGaOₓ | 48.7 | 31.3 | 20.0 | 33.4 | 108 | 24.6 | 76.7 | 214.0 | 3.6 | 0.661 |
| 60CuZnGaOₓ | 65.5 | 21.0 | 13.5 | 31.9 | 176 | 14.3 | 58.4 | 110.5 | 6.7 | — |
| 43CuZnOₓ | 43.7 | 56.3 | 0 | 3.6 | 423 | 12.6 | 31.7 | 85.4 | 8.0 | 0.514 |
| 43CuZnGaOₓ(3:2) | 47.6 | 36.8 | 15.6 | 26.5 | 128 | 29.1 | 75.9 | 210.2 | 3.6 | — |
| 43CuZnGaOₓ(1:3) | 31.7 | 16.6 | 31.7 | 17.1 | 82 | 22.2 | 55.2 | 156.5 | 5.3 | — |
| 43CuGaOₓ | 54.9 | 0 | 45.1 | 15.7 | 95 | 9.3 | 25.9 | 74.6 | 11.7 | 1.102 |

From the table above it can be seen that the surface area of Cu in the catalyst (SA_{cat}) is an important factor in determining MeOH conversion. Therefore it is necessary to understand the factors that determine SA_{cat}, in order to be able to rationally design more effective SRM catalysts in the future.

FIG. 35 shows a contour map of how SA_{cat} varies with the Cu/Zn/Ga content of the catalyst. There is a clear ‘hotspot’ at around 50% Cu, 30% Zn and 20% Ga where SA_{cat} is very high. Deviations from this formulation in any direction lead to a reduced specific surface area of Cu. As Cu content increases we would expect SA_{cat} to increase as well, but we observe that beyond ˜50% Cu loading the surface area starts to decrease again. This is due to Cu sintering; there is now so much Cu in the material that the Zn/Ga oxide can no longer keep the particles effectively dispersed, and so they aggregate together upon calcination leading to increased particle size and reduced surface area. It was seen from the powder XRD that calcined 43CuZnGaOₓ contained a cubic spinel phase that was not present in either 43CuZnOₓ or 43CuGaOₓ. It is therefore likely that this spinel phase is stabilising Cu in a highly dispersed state, leading to a higher surface area.

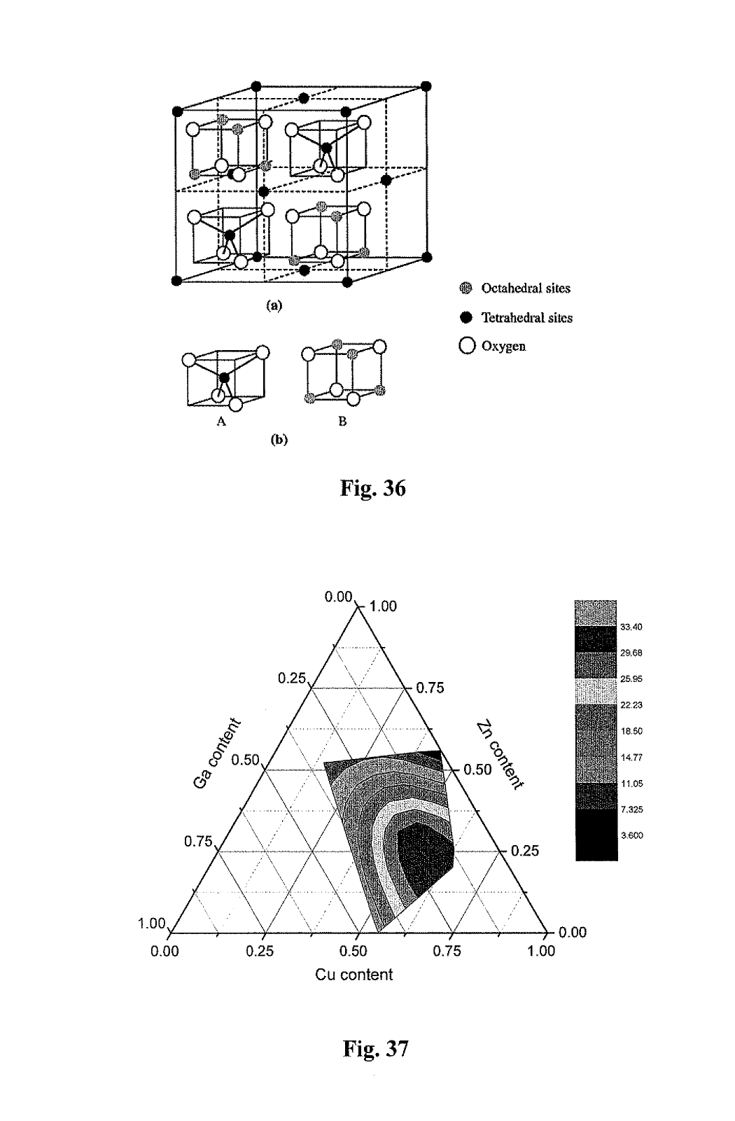

The spinel structure consists of a cubic close-packed oxide with general formula AB₂O₄ containing one Oₕ site and two T_{d} sites per oxide (FIG. 36). In a normal spinel, A exists as A²⁺ ions occupying ⅛ of the T_{d} holes whilst B exists as B³⁺ ions occupying the Oₕ holes. In an inverse spinel, A²⁺ instead occupies ½ the Oₕ holes due to LFSE considerations and B³⁺ occupies ½ Oₕ holes and ⅛ T_{d} holes. In the case of a spinel composed of Cu, Zn and Ga, the structure will most likely exist in the inverse form due to LFSE considerations, with Cu²⁺ (d⁹ configuration) occupying the Oₕ environment and Ga³⁺ ions (d¹⁰) in the T_{d} environment. Zn²⁺ (d¹⁰) has no LFSE preference for the Oₕ site, and so can exist on either the T_{d} or Oₕ sites. Cu, Zn and Ga are located adjacent to each other on the periodic table, and their ionic radii are all similar, especially Cu and Zn(Cu²⁺=8.7 nm, Zn²⁺=8.8 nm, Ga³⁺=7.6 nm), therefore it is expected that there will be a large degree of site swapping and substitution between Cu²⁺ and Zn²⁺ within the spinel lattice. Non-stoichiometric spinels (NSS) containing Cu have been reported in the literature. It was found that spinels containing excess Cu were able to accommodate the extra Cu ions as interstitial Cu⁺ within the spinel lattice. The mechanism for the formation of interstitial Cu⁺ is proposed as occurring via loss of oxygen, as follows (Eq. L):

CuGa₂O₄⇄Cu₁₋₂ₓ(Cuᵢ⁺)₂ₓGa₂O₄₋ₓ+½xO₂ (Eq. L)

Excess Cu²⁺ from CuO may then enter the spinel phase to fill the vacant lattice site, forming a non-stoichiometric phase. Cu²⁺ (d⁹) in an Oₕ environment exhibits the Jahn-Teller effect, causing elongation and hence weakening of the Cu—O bonds along the axial plane. This axial distortion is what drives the formation of the tetragonal spinel phase observed in the case of CuGa₂O₄. However, partially substituting Cu²⁺ for Zn²⁺ (d¹⁰) helps to remove the instability caused by the Jahn-Teller effect, therefore stabilising the material in the cubic NSS phase observed for 43CuZnGaOₓ. It was shown from the TPR that for Cu/Zn/Ga oxide materials, Cu can exist in three separate environments with different reducibilities. The low temperature reduction site, αCu, appears to correspond with Cu⁺ existing on or near the surface of the material. This environment is identified as interstitial Cu⁺ ions within the NSS phase, which will be easily reducible to Cu metal. In the case of 43CuGaOₓ, which consists of poorly crystalline tetragonal CuGa₂O₄, the spinel structure cannot be effectively maintained upon the reduction of interstitial Cu⁺, leading to significant Cu aggregation and poor activity. However, in the case of 43CuZnGaOₓ, the presence of Zn allows the structure to maintain its cubic NSS structure upon reduction, leading to high Cu dispersion and surface area. It is this αCu site that is critical to the activity of the catalyst, since the steam reforming is taking place at 195° C., therefore βCu and γCu will not be reduced under the reaction conditions. It was observed that 43CuZnGaOₓ displayed a broad EPR signal not seen in either 43CuGaOₓ or 43CuZnOₓ, and it was also seen from TPR that neither 43CuGaOₓ nor 43CuZnOₓ displayed a reduction peak corresponding with the βCu environment. We can therefore conclude that βCu corresponds with the Cu²⁺ ions existing within the cubic NSS phase. The γCu environment, which displayed the strongest TPR peak with 43CuGaOₓ, corresponds with Cu²⁺ ions in the tetragonal NSS phase. This tetragonal spinel phase was shown from XRD and EPR to also be present in 43CuZnGaOₓ to some extent. However, no γCu TPR peak was observed for 43CuZnGaOₓ. It may be the case that this peak was obscured by the larger βCu peak, since it was observed that both 43CuZn3Ga2Oₓ and 43CuZn1Ga3Oₓ contained a γCu peak in the TPR. The αCu environment, which was observed in the TPR for all the Ga-containing catalysts, does not give an EPR signal because Cu⁺ (d¹⁰) is not paramagnetic.

FIG. 37 is a contour map showing how MeOH conversion varies with atomic content. Here we can see the close correlation between the MeOH conversion and SA_{cat}, since once again there is a hotspot around 50% Cu, 30% Zn and 20% Ga. Without wishing to be bound by theory, it is thought that this is because a higher Cu surface area gives rise to a greater number of available active sites. A factor influencing the location of the hotspot may be the stability of the cubic NSS phase due to the presence of Zn²⁺, leading to the facile reduction of interstitial Cu⁺ ions to produce highly dispersed Cu⁰ particles on the surface.

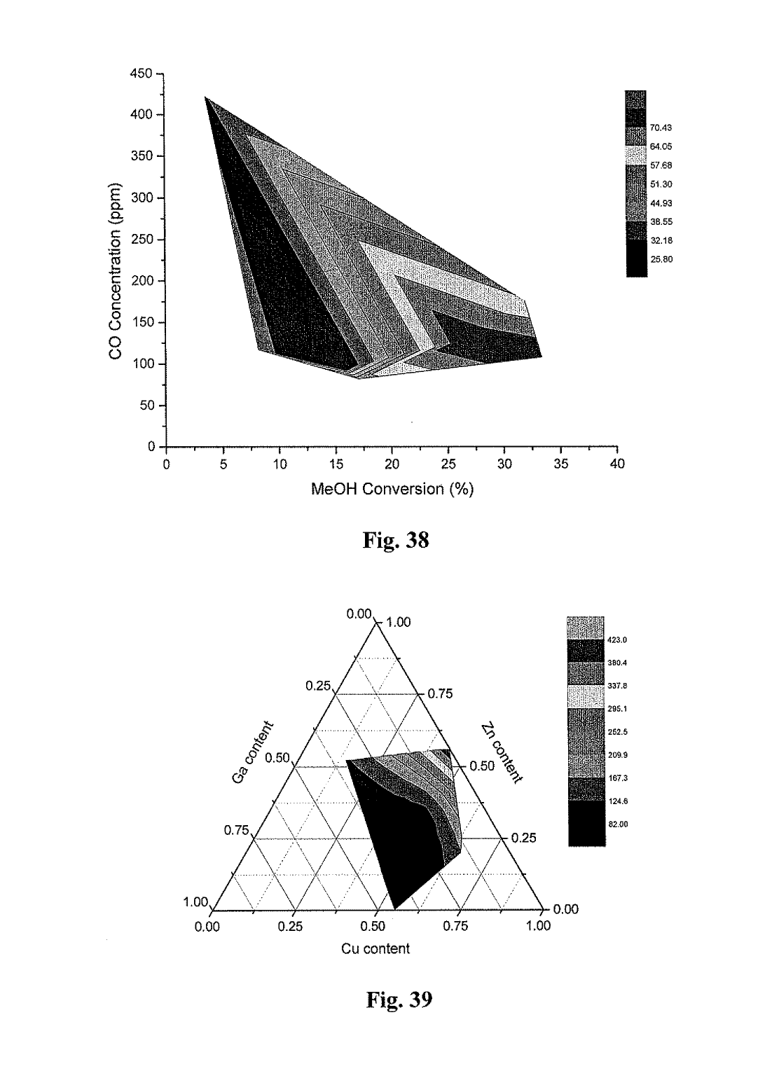

FIG. 38 is a contour plot showing how SA_{cat} affects MeOH conversion and CO production. This graph demonstrates that, in general, CO production decreases as MeOH conversion increases, which is in turn dependent on the surface area of Cu in the catalyst, Therefore it would appear that CO production could be minimised by maximising SA_{cat}. Without wishing to be bound by theory, this would imply that the Zn/Ga oxide support plays no active role in the catalytic process itself, and exists only to stabilise a highly dispersed, reducible form of Cu. However, FIG. 39, which shows how CO production varies with the catalyst formulation, shows that this is not necessarily the case. The contour map in FIG. 39 seems to show that CO production is not only related to SA_{cat}, but also strongly related to Ga content, with CO levels decreasing as the Ga content is increased. This is an important discovery, as it means that Ga in the support is playing an active role in the catalytic process by somehow suppressing CO formation. We have seen from AC impedance that Ga in the support leads to decreasing conductivity and increasing thermal activation energy. This is because as the Ga content increases, the poorly crystalline tetragonal NSS phase begins to predominate, which has reduced Cu mobility due to its lack of ordered crystallinity. The oxygen mobility will also be reduced for the same reason. O-vacancies are believed to play a key role in CO formation; therefore the lack of available O-vacancies in the support due to the poorly crystalline nature of the tetragonal NSS phase, caused by the abundance of Ga, would explain the downward trend in CO formation as Ga content is increased. There is, therefore, a balance to be struck between maximising the αCu surface area in order to promote MeOH conversion, and maximising the Ga content so as to suppress CO formation. This balance is struck at 50% Cu, 30% Zn and 20% Ga, where the atomic ratio is just right that the cubic NSS phase is stabilised, leading to a high surface area upon reduction of the highly reducible interstitial Cu⁺ ions, but the O-mobility in the bulk support is sufficiently reduced that CO production is effectively suppressed.

### Mechanism

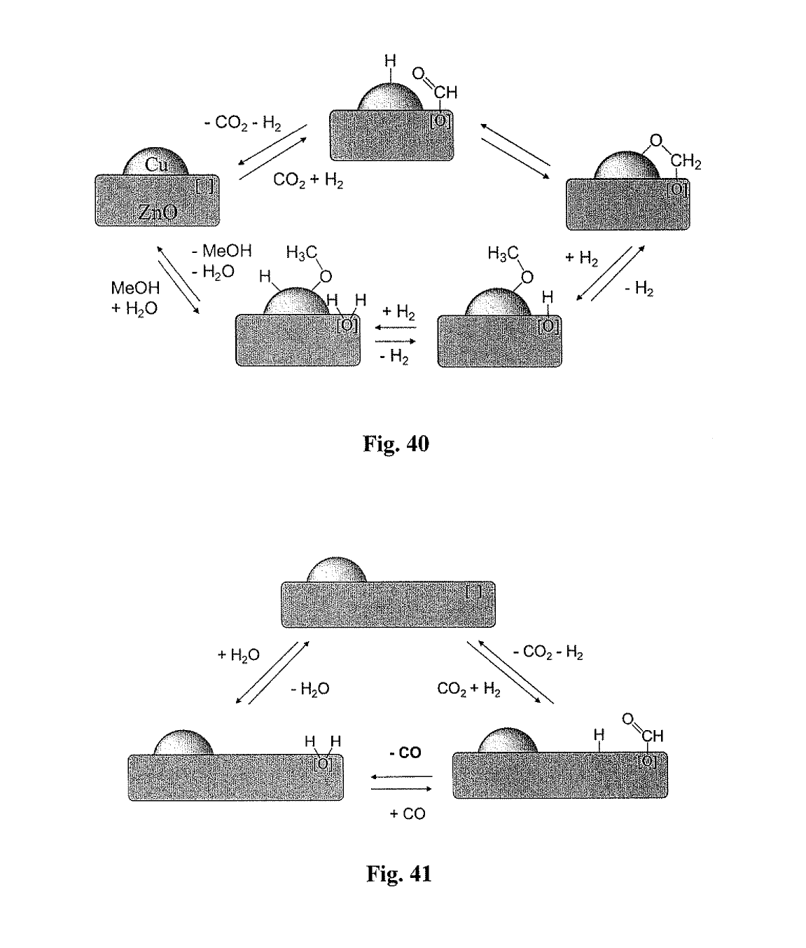

Oxygen vacancies are believed to play an important role in the steam reforming cycle. FIG. 40 shows a possible mechanism for the reversible process with Cu metal on a ZnO support. Other work in our group has shown that the greater metal-support interaction between the Cu and plate-like ZnO than rod-like ZnO leads to higher activity in the methanol formation reaction (clockwise process). It has been demonstrated that the metal-support interaction leads to an electron transfer from the support to the Cu, leading to a greater number of O-vacancies at the metal-support interface. These vacancies are hypothesised to play an important role in the catalytic cycle by providing an adsorption site for CO₂ adjacent to Cu, to which hydrogen can add to produce the adsorbed formate species. Upon further addition of hydrogen, the adsorbed species rearranges itself onto the Cu surface, leaving oxygen behind in the support, which then releases MeOH+H₂O upon further H₂ addition. The same process can take place in reverse (anti-clockwise) for the steam reforming reaction. Methanol adsorbs onto the Cu surface, and H₂O adsorbs into an O-vacancy site in the support. The adsorbed methanol species picks up oxygen from the support and is released as CO₂. The water constantly replenishes the oxygen supply.

CO formation is supposedly produced exclusively as a secondary product, via the reverse WGS reaction. FIG. 41 shows a possible catalytic cycle for the process. The reverse WGS reaction (clockwise process) takes place when CO₂, produced via SRM, is adsorbed into an O-vacancy site in the support that is not at the metal-support interface. It therefore cannot undergo the necessary steps to produce MeOH, and so instead is either re-released as CO₂ or loses oxygen in the presence of hydrogen to produce CO+H₂O.

Therefore, in order to maximise CO₂ formation and minimise CO formation, the Cu dispersion and surface area should be as high as possible, to maximise the possibility that an adsorbed species will be at the metal-support interface. We have observed herein that MeOH conversion is strongly related to SA_{cat}, and that maximum conversion occurred for 43CuZnGaOₓ, which contains highly dispersed, reducible αCu, which upon reduction will exist as very small Cu metal particles on the surface, whilst the CuZnGaOₓ cubic NSS phase is retained beneath. The reduction of the interstitial Cu⁺ will lead to the production of O-vacancies at the metal support interface, thus providing the active sites required for SRM (Eq. M):

$$\{ ( {Cu}_{i}^{+} ) {2}_{} ( {O}^{2 -} ) \} + x {H}_{2} \to \{ ( {Cu}_{i}^{+} ) {2 - x}_{} {( {O}^{2 -} )}_{1 - x} \} + 2 {x Cu}^{0} + x {H}_{2} O \qquad ( Eq . M )$$

Another way to prevent CO formation would be to minimise the number of O-vacancies that are not in the vicinity of Cu. We observed earlier that the addition of Ga suppresses CO formation, which was explained by the reduction in O-mobility caused by the increasing formation of a poorly crystalline tetragonal spinel phase. Therefore, the Zn/Ga support is playing a dual role; not only does it encourage the formation of the cubic NSS phase, leading to a highly dispersed, highly reducible form of Cu, it also provides enough O-vacancies at the metal-support interface that the steam reforming process can proceed effectively, but few available O-vacancies in the bulk support, so that CO production via the reverse WGS reaction is effectively suppressed.

### Summary

In this Example it has been shown that the enhanced performance of 43CuZnGaOₓ is due to the formation of a stable cubic NSS phase containing highly reducible, well-dispersed interstitial Cu⁺ ions, and that the presence of Ga effectively reduces the availability of O-vacancies in the bulk support leading to suppression of CO formation. αCu was identified as interstitial Cu ions in the NSS phases, βCu as Oₕ Cu²⁺ in the cubic NSS phase and γCu as Oₕ Cu²⁺ in the poorly crystalline tetragonal NSS phase. 43CuZnOₓ exists as a Cu/ZnO solid solution containing highly mobile T_{d} Cu²⁺ ions. The presence of Zn in 43CuZnGaOₓ stabilised the cubic NSS phase, leading to enhanced Cu dispersion and surface area upon reduction of αCu. 43CuGaOₓ, although containing reducible αCu, existed as a poorly crystalline tetragonal NSS phase, and was therefore unable to maintain high Cu dispersion upon H₂ reduction, leading to poor MeOH conversion. In 43CuZnOₓ, the combination of low metal reducibility and high O-mobility led to the low MeOH conversion and high CO production observed. In 43CuGaOₓ the low O-mobility helped suppress CO formation, but the aggregation of Cu diminished the MeOH conversion. In 43CuZnGaOₓ the reduction of αCu led to the formation of O-vacancies at the metal-support interface, but the availability of O-vacancies in the bulk support was reduced due to the high Cu dispersion, leading to the suppression of CO formation.

It has been shown that 43CuZnGaOₓ can achieve H₂ productivity of 393.6 ml g⁻¹ cat. hr⁻¹ with <10 ppm CO formation at 150° C. By demonstrating here that CO production can be suppressed to <10 ppm whilst maintaining significant H₂ production, the invention has provided commercially viable low temperature SRM catalysts for use in PEM fuel cells. Future work will involve the optimisation of the catalyst SRM.

In conclusion, a series of Cu/Zn/Ga mixed metal oxide solid catalysts were prepared via a co-precipitation method and tested for their activity in the low temperature steam reforming of methanol (195° C., 0.1 mil min′ reactant flow rate, 1:2 methanol/water ratio). 43CuZnGaOₓ containing ˜50% Cu, 30% Zn and 20% Ga was found to be the most active catalyst, displaying high activity combined with very little CO production. This can be explained in terms of the catalyst structure, a heterogeneous mixture of various phases including a non-stoichiometric cubic spinel phase, stabilised by the presence of Zn²⁺, containing excess interstitial Cu⁺ ions that were highly dispersed and highly reducible, leading to an enhanced surface area of Cu in the catalyst. The suppression of CO formation was explained in terms of the mechanism, whereby the high Cu dispersion led to an abundance of O-vacancy active sites at the metal-support interface for the steam reforming process, but few O-vacancies available in the bulk support for the reverse WGS reaction.

### Example 3

### CO₂ Hydrogenation to Methanol

TABLE 10
Results for CO₂ hydrogenation to methanol: comparing
catalysts of the invention with an industrial catalyst

| | CH3OH | CO | Conver- | Select- | Carbon |
|---|---|---|---|---|---|
| Sample | % Yield | % Yield | sion/% | ivity/% | balance/% |
| 43% Cu—ZnGaOₓ | 25.5 | 7.5 | 33.0 | 77.2 | 89.6 |
| (1^{st}) | | | | | |
| 43% Cu—ZnGaOₓ | 25.7 | 8.3 | 34.0 | 75.5 | 90.7 |
| (2^{nd}) | | | | | |
| JM HiFUELT | 24.2 | 8.4 | 32.6 | 74.3 | 92.3 |
| R120 | | | | | |

1^{st} and 2^{nd} correspond to 1^{st} testing and 2^{nd} testing
Weight used: 0.2 g
Composition of gas feeds: H₂/CO₂ = 3:1
Pressure: 5 Mpa
Temperature: 503K
Flow rate: 25 ml/min
GC analysis of products after the initial 2-3 hs
The Industrial catalyst (JM-HiFUELT R120 catalyst) was tested under 513K for comparison.

TABLE 11
Results for the reverse reaction: steam reformation
of methanol to H₂/CO₂:comparing catalysts of the
invention with an industrial catalyst

| | MeOH | CO Concen- |
|---|---|---|
| Catalyst | Conversion (%) | tration (ppm) |
| JM-HiFUELT | 29.3 | 251 |
| R120 catalyst | | |
| 43% Cu—ZnGaOx | 33.4 | 108 |

Weight used: 0.40 g + 0.40 g silicon carbide
Liquid feeds: CH₃OH:H₂O molar ratio = 1:2
Liquid flow rate of 0.1 ml min⁻¹ and then mix with 10 ml min⁻¹ of N₂ gas
Pressure: 101.3 Kpa
Temperature: 468K
GC analysis of products after the initial 2-3 hs

1. A process for producing H₂ by steam reforming of methanol, which process comprises contacting a gas phase comprising (a) CH₃OH and (b) H₂O with a solid catalyst, which solid catalyst comprises a mixed metal oxide, which mixed metal oxide comprises copper, zinc and gallium, wherein the atomic percentage of copper relative to the total number of metal atoms in the oxide is from 20 at. % to 55 at. %.

2. (canceled)

3. A process according to claim 1 wherein said atomic percentage of copper is from 35 at. % to 55 at. %.

4. (canceled)

5. A process according to claim 1 wherein the atomic percentage of gallium relative to the total number of metal atoms in the oxide is equal to or greater than 5 at. %.

6-7. (canceled)

8. A process according to claim 1 wherein the atomic percentage of gallium relative to the total number of metal atoms in the oxide is from 10 at. % to 40 at. %.

9-10. (canceled)

11. A process according to claim 1 wherein the atomic percentage of zinc relative to the total number of metal atoms in the oxide is from 10 at. % to 50 at. %.

12. (canceled)

13. A process according to claim 1 wherein the atomic percentages of copper, zinc and gallium relative to the total number of metal atoms in the oxide are from 30 to 55 at. % copper, at least 15 at. % zinc, and at least 10 at. % gallium.

14-15. (canceled)

16. A process according to claim 1 wherein the atomic percentages of copper, zinc and gallium relative to the total number of metal atoms in the oxide are from 30 to 55 at. % copper, from 15 to 45 at. % zinc, and from 10 to 40 at. % gallium.

17-19. (canceled)

20. A process according to claim 1 wherein the metal atoms in the mixed metal oxide consist of said copper, zinc and gallium, wherein the metal atoms in the mixed metal oxide consist of x at. % copper, y at. % gallium, and z at. % zinc, wherein x is from 30 to 55, y is from 10 to 40, and z is 100−(x+y).

21-25. (canceled)

26. A process according to claim 1 wherein the mixed metal oxide has a specific copper metal surface area, as measured by N₂O chemisorption, of at least 50 m²/g-catalyst, or wherein the mixed metal oxide has a specific copper metal surface area per gram copper, as measured by N₂O chemisorption, of at least 150 m²/g-Cu.

27-29. (canceled)

30. A process according to claim 1 wherein the mixed metal oxide comprises particles of said copper wherein the copper particles have a mean particle size of less than or equal to 10 nm, and/or wherein the mixed metal oxide has a copper metal dispersion of at least 15%.

31-32. (canceled)

33. A process according to claim 1 wherein the mixed metal oxide comprises a non-stoichiometric spinel phase comprising copper, zinc and gallium.

34. A process according to claim 33 wherein the spinel phase comprises octahedral Cu²⁺ and interstitial Cu⁺.

35. A process according to claim 33 wherein the spinel phase comprises particles of copper metal, wherein the copper particles have a mean particle size of less than or equal to 10 nm.

36. A process according to claim 1 wherein the catalyst further comprises a solid support material.

37. A process according to claim 1 wherein the catalyst is obtained by a process as defined in claim 56.

38. A process according to claim 1 wherein the step of contacting the gas phase with the solid catalyst is performed at a temperature which does not exceed 200° C., and at atmospheric pressure.

39-41. (canceled)

42. A process according to claim 1 which occurs substantially without any formation of carbon monoxide.

43. A process according to claim 1 wherein the level of CO produced does not exceed 100 ppm.

44. (canceled)

45. A process according claim 1 wherein the percent conversion of methanol is at least 20%.

46. A process according to claim 1 wherein the molar ratio of H₂O to CH₃OH in said gas phase is equal to or greater than 1.

47-50. (canceled)

51. A process according to claim 1 which further comprises recovering said H₂.

52. A process according to claim 1 which further comprises using the H₂ produced as a fuel, or using the H₂ produced to power a fuel cell.

53. (canceled)

54. A catalyst for use in a process for producing H₂ by steam reforming of methanol, which catalyst comprises a mixed metal oxide, which mixed metal oxide comprises copper, zinc and gallium, wherein the atomic percentage of copper relative to the total number of metal atoms in the oxide is from 20 at. % to 55 at. %.

55. A catalyst according to claim 54, wherein the atomic percentages of copper, zinc and gallium relative to the total number of metal atoms in the oxide are from 30 to 55 at. % copper, at least 15 at. % zinc, and at least 10 at. % gallium.

56. A process for producing a catalyst, which catalyst is suitable for use in a process for producing H₂ by steam reforming of methanol, and which catalyst comprises a mixed metal oxide, which mixed metal oxide comprises copper, zinc and gallium, wherein the atomic percentage of copper relative to the total number of metal atoms in the oxide is from 20 at. % to 55 at. %, which process comprises:
(1) a co-precipitation step, comprising contacting: (a) a solution of copper nitrate, zinc nitrate and gallium nitrate, wherein the atomic percentage of copper relative to the total number of metal atoms in said solution is from 20 at. % to 55 at. %, with (b) a metal carbonate, to produce a co-precipitate comprising said copper, zinc and gallium;
(2) a separation step, comprising separating the co-precipitate from solution; and
(3) a calcination step, comprising calcining the co-precipitate by heating the co-precipitate in air.

57. A process according to claim 56 which further comprises: (4) a reduction step, comprising heating the calcined product in the presence of H₂.

58-59. (canceled)

60. A process according to claim 56 wherein the catalyst is as defined in claim 13 and the atomic percentages of copper, zinc and gallium in said solution of copper nitrate, zinc nitrate and gallium nitrate, relative to the total number of metal atoms in said solution, are the same as the atomic percentages of copper, zinc and gallium in said catalyst as defined in claim 13.

61-68. (canceled)

69. A process according to claim 56, further comprising recovering the catalyst and using it in a process as defined in claim 1 for producing H₂ by steam reforming of methanol.

70-72. (canceled)

73. A fuel cell system comprising a fuel cell and a methanol reformer which methanol reformer comprises a catalyst as defined in claim 54.

74. A fuel cell system according to claim 73 wherein the fuel cell is a proton exchange membrane (PEM) fuel cell.

75. A portable electronic device comprising a fuel cell system according to claim 74.

76-77. (canceled)

78. A process for producing methanol by the hydrogenation of carbon dioxide, which process comprises contacting a gas phase comprising (a) CO₂ and (b) H₂, with a solid catalyst, which solid catalyst comprises a mixed metal oxide, which mixed metal oxide comprises copper, zinc and gallium, wherein the atomic percentage of copper relative to the total number of metal atoms in the oxide is from 20 at. % to 55 at. %.

79. A process according to claim 78 wherein the atomic percentages of copper, zinc and gallium relative to the total number of metal atoms in the oxide are from 30 to 55 at. % copper, at least 15 at. % zinc, and at least 10 at. % gallium.

80. A process according to claim 78 wherein the catalyst is obtained by a process as defined in claim 56.

81. A process according to claim 78 which further comprises recovering said methanol.
